(19) United States
(12) Patent Application Publication
(10) Pub. No.: US 2025/0387009 A1
(43) Pub. Date: Dec. 25, 2025

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: Kentaro OSHIRO, Kanagawa (JP)

(73) Assignee: FUJIFILM Corporation, Tokyo (JP)

(21) Appl. No.: 19/315,703

(22) Filed: Sep. 1, 2025

Related U.S. Application Data
(63) Continuation of application No. PCT/JP2024/005564, filed on Feb. 16, 2024.

(30) Foreign Application Priority Data
Mar. 15, 2023 (JP) 2023-041360

(51) Int. Cl. A61B 1/00

(52) U.S. Cl. CPC A61B 1/000094; A61B 1/000096; A61B 1/00048; A61B 1/0005

(57) ABSTRACT

A medical support device includes a processor. The processor is configured to acquire a size of an observation target region appearing in a medical image obtained by imaging an imaging target region including the observation target region with a modality. The processor is configured to output auxiliary information for assisting in clinical decision making in cases in which the size falls within a size range determined with respect to a reference value for the decision making.

### CROSS-REFERENCE TO RELATED APPLICATIONS

This application is a continuation application of International Application No. PCT/JP2024/005564, filed Feb. 16, 2024, the disclosure of which is incorporated herein by reference in its entirety. Further, this application claims priority from Japanese Patent Application No. 2023-041360, filed Mar. 15, 2023, the disclosure of which is incorporated herein by reference in its entirety.

### BACKGROUND

### 1. Technical Field

The technology of the present disclosure relates to a medical support device, an endoscope system, a medical support method, and a program.

### 2. Related Art

WO2020/110214A discloses an endoscope system including an image input unit, a lesion detection unit, an oversight risk analysis unit, a notification control unit, and a notification unit.

In the endoscope system described in WO2020/110214A, a plurality of observation images obtained by imaging a photographic subject with an endoscope are sequentially input to the image input unit. The lesion detection unit detects a lesion area to be observed with the endoscope from the observation images.

The oversight risk analysis unit determines a degree of oversight risk, which is a risk of an operator overlooking the lesion area, based on the observation images. The notification control unit controls a notification means and a notification method of detection of the lesion area, based on the degree of oversight risk. The notification unit notifies the operator of the detection of the lesion area under the control of the notification control unit.

In the endoscope system described in WO2020/110214A, the oversight risk analysis unit includes a lesion analysis unit that analyzes the oversight risk based on the state of the lesion area. The lesion analysis unit includes a lesion size analysis unit that estimates the size of the lesion area itself.

In the endoscope system described in WO2020/110214A, the notification control unit performs notification control to generate a marker image indicating the lesion area and superimpose the marker image on the observation images, and changes at least one of the color, thickness, or size of the marker image in accordance with the degree of the risk of the lesion area.

JP2022-535873A discloses a technique for presenting a GUI for dynamically tracking at least one polyp in an endoscopic image, involving computing a dimension of the polyp and presenting an alert within the GUI when the dimension of the polyp exceeds a threshold value.

### SUMMARY

An embodiment according to the technology of the present disclosure provides a medical support device, an endoscope system, a medical support method, and a program that can contribute to improvement in the accuracy of clinical decision making.

A first aspect according to the technology of the present disclosure is a medical support device including a processor, the processor being configured to acquire a size of an observation target region appearing in a medical image obtained by imaging an imaging target region including the observation target region with a modality; and output auxiliary information for assisting in clinical decision making in cases in which the size falls within a size range determined with respect to a reference value for the decision making.

A second aspect according to the technology of the present disclosure is the medical support device according to the first aspect, in which the size is measured based on the medical image.

A third aspect according to the technology of the present disclosure is the medical support device according to the first aspect or the second aspect, in which the auxiliary information includes the size of the observation target region in at least one direction; position information enabling identification of a position of the observation target region in the medical image; shape information enabling identification of a shape of the observation target region; measurement direction information enabling identification of a measurement direction used to measure the size; a degree of certainty obtained from AI in cases in which the size is measured using the AI; size variation range information enabling identification of a range of variation in the size identified in cases in which the size is measured based on a plurality of the medical images; an image indicating the observation target region; and/or a statistical measure based on previously measured size.

A fourth aspect according to the technology of the present disclosure is the medical support device according to any one of the first to third aspects, in which the reference value and/or the size range is determined based on medical knowledge.

A fifth aspect according to the technology of the present disclosure is the medical support device according to any one of the first to fourth aspects, in which the reference value and/or the size range is determined based on a characteristic of the observation target region.

A sixth aspect according to the technology of the present disclosure is the medical support device according to any one of the first to fifth aspects, in which the reference value and/or the size range is determined in accordance with a given instruction.

A seventh aspect according to the technology of the present disclosure is the medical support device according to any one of the first to sixth aspects, in which the auxiliary information is output by displaying the auxiliary information on a screen.

An eighth aspect according to the technology of the present disclosure is the medical support device according to any one of the first to seventh aspects, in which the auxiliary information is output by displaying the auxiliary information on a first screen, the medical image is displayed on a second screen different from the first screen, and the first screen and the second screen are arranged in a manner that enables comparison with each other.

A ninth aspect according to the technology of the present disclosure is the medical support device according to any one of the first to eighth aspects, in which the decision making is decision making as to whether to excise the observation target region from the imaging target region.

A tenth aspect according to the technology of the present disclosure is the medical support device according to any one of the first to ninth aspects, in which the modality is an endoscope system.

An eleventh aspect according to the technology of the present disclosure is the medical support device according to any one of the first to tenth aspects, in which the medical image is an endoscopic image obtained by imaging the imaging target region with an endoscope.

A twelfth aspect according to the technology of the present disclosure is the medical support device according to any one of the first to eleventh aspects, in which the observation target region is a lesion.

A thirteenth aspect according to the technology of the present disclosure is an endoscope system including the medical support device according to any one of the first to twelfth aspects, and an endoscope that performs imaging of the imaging target region.

A fourteenth aspect according to the technology of the present disclosure is a medical support method including acquiring a size of an observation target region appearing in a medical image obtained by imaging an imaging target region including the observation target region with a modality; and outputting auxiliary information for assisting in clinical decision making in cases in which the size falls within a size range determined with respect to a reference value for the decision making.

A fifteenth aspect according to the technology of the present disclosure is the medical support method according to the fourteenth aspect, in which the modality includes an endoscope, and the medical support method includes using the endoscope.

A sixteenth aspect according to the technology of the present disclosure is a program for causing a computer to execute a medical support process, the medical support process including acquiring a size of an observation target region appearing in a medical image obtained by imaging an imaging target region including the observation target region with a modality; and outputting auxiliary information for assisting in clinical decision making in cases in which the size falls within a size range determined with respect to a reference value for the decision making.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope system is used;

FIG. 2 is a conceptual diagram illustrating an example overall configuration of the endoscope system;

FIG. 3 is a block diagram illustrating an example hardware configuration of an electric system of the endoscope system;

FIG. 4 is a block diagram illustrating an example of functions of main components, according to an embodiment, of a processor included in a medical support device, and an example of information stored in an NVM;

FIG. 5 is a conceptual diagram illustrating an example of the content of a process of a recognition unit and a control unit;

FIG. 6 is a conceptual diagram illustrating an example of the content of a process of a measurement unit;

FIG. 7 is a conceptual diagram illustrating an example of an aspect in which a plurality of previous sizes are stored in a size storage area;

FIG. 8 is a conceptual diagram illustrating an example of the content of a process of the control unit;

FIG. 9 is a conceptual diagram illustrating an example of the content of a process of the control unit and an example of the content displayed on a screen in cases in which first display control is to be performed;

FIG. 10 is a conceptual diagram illustrating an example of the content of a process of the control unit and an example of the content displayed on the screen in cases in which second display control is to be performed;

FIG. 11 is a flowchart illustrating an example of the flow of a medical support process;

FIG. 12 is a conceptual diagram illustrating a first modification of auxiliary information displayed in a second display region;

FIG. 13 is a conceptual diagram illustrating a second modification of the auxiliary information displayed in the second display region;

FIG. 14 is a conceptual diagram illustrating a modification of a method for determining a reference value;

FIG. 15 is a conceptual diagram illustrating a modification of a method for determining a reference size range;

FIG. 16 is a conceptual diagram illustrating an example of the content of a process for deriving a reference value from characteristic information;

FIG. 17 is a conceptual diagram illustrating an example of the content of a process for deriving a reference size range from characteristic information;

FIG. 18 is a conceptual diagram illustrating examples of a destination to which various kinds of information are to be output; and

FIG. 19 is a conceptual diagram illustrating an example of a series of processing operations in which a processor of the endoscope system provides a process execution request to an external device via a network, the external device executes a process in response to the process execution request, and the processor of the endoscope system receives a process result from the external device.

### DESCRIPTION OF THE PREFERRED EMBODIMENTS

An example of an embodiment of a medical support device, an endoscope system, a medical support method, and a program according to the technology of the present disclosure will be described hereinafter with reference to the accompanying drawings.

First, terms used in the following description will be described.

CPU is an abbreviation for “Central Processing Unit”. GPU is an abbreviation for “Graphics Processing Unit”. RAM is an abbreviation for “Random Access Memory”. NVM is an abbreviation for “Non-volatile memory”. EEPROM is an abbreviation for “Electrically Erasable Programmable Read-Only Memory”. ASIC is an abbreviation for “Application Specific Integrated Circuit”. PLD is an abbreviation for “Programmable Logic Device”. FPGA is an abbreviation for “Field-Programmable Gate Array”. SoC is an abbreviation for “System-on-a-chip”. SSD is an abbreviation for “Solid State Drive”. USB is an abbreviation for “Universal Serial Bus”. HDD is an abbreviation for “Hard Disk Drive”. EL is an abbreviation for “Electro-Luminescence”. CMOS is an abbreviation for “Complementary Metal Oxide Semiconductor”. CCD is an abbreviation for “Charge Coupled Device”. AI is an abbreviation for “Artificial Intelligence”. BLI is an abbreviation for “Blue Light Imaging”. LCI is an abbreviation for “Linked Color Imaging”. I/F is an abbreviation for “Interface”. SSL is an abbreviation for “Sessile Serrated Lesion”. LAN is an abbreviation for “Local Area Network”. WAN is an abbreviation for “Wide Area Network”. FIFO is an abbreviation for “First In First Out”.

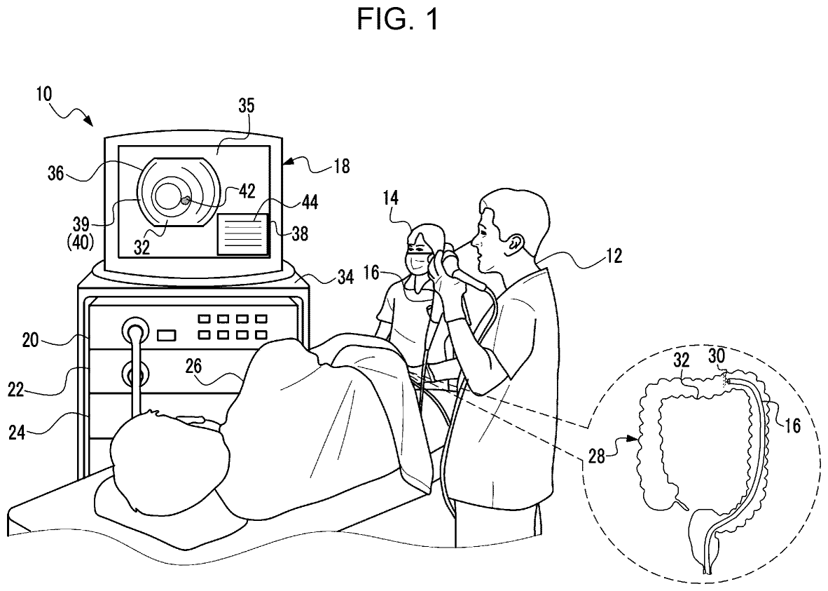

As an example, as illustrated in FIG. 1, an endoscope system 10 is used by a doctor 12 in an endoscopic examination or the like. The endoscopic examination is assisted by staff such as a nurse 14. In the present embodiment, the endoscope system 10 is an example of the “modality” and the “endoscope system” according to the technology of the present disclosure.

The endoscope system 10 is connected to a communication device (not illustrated) in a communicable manner, and information obtained by the endoscope system 10 is transmitted to the communication device. An example of the communication device is a server and/or a client terminal (for example, a personal computer, a tablet terminal, and/or the like) that manages various kinds of information such as electronic medical records. The communication device receives the information transmitted from the endoscope system 10 and executes a process using the received information (for example, a process of storing the information in an electronic medical record or the like).

The endoscope system 10 includes an endoscope 16, a display device 18, a light source device 20, a control device 22, and a medical support device 24. In the present embodiment, the endoscope 16 is an example of the “endoscope” according to the technology of the present disclosure.

The endoscope system 10 is a modality for performing medical care for a large intestine 28 included in the body of a subject 26 (for example, a patient) using the endoscope 16. In the present embodiment, the large intestine 28 is a target to be observed by the doctor 12.

The endoscope 16 is used by the doctor 12 and is inserted into a body cavity of the subject 26. In the present embodiment, the endoscope 16 is inserted into the large intestine 28 of the subject 26. The endoscope system 10 causes the endoscope 16 inserted into the large intestine 28 of the subject 26 to perform imaging of the inside of the large intestine 28 of the subject 26, and performs various medical treatments on the large intestine 28 as necessary.

The endoscope system 10 performs imaging of the inside of the large intestine 28 of the subject 26 to acquire an image indicating the state of the inside of the large intestine 28, and outputs the acquired image. In the present embodiment, the endoscope system 10 is an endoscope having an optical imaging function of capturing an image of reflected light obtained by irradiating the inside of the large intestine 28 with light 30 and reflecting the light 30 from an intestinal wall 32 of the large intestine 28.

While the endoscopic examination of the large intestine 28 is exemplified here, this is merely an example, and the technology of the present disclosure is also applicable to an endoscopic examination of a luminal organ such as the esophagus, the stomach, the duodenum, or the trachea.

The light source device 20, the control device 22, and the medical support device 24 are installed in a cart 34. The cart 34 is provided with a plurality of shelves along the vertical direction, and the medical support device 24, the control device 22, and the light source device 20 are installed on the shelves from bottom to top. The display device 18 is installed on top of the cart 34.

The control device 22 performs overall control of the endoscope system 10. Under the control of the control device 22, the medical support device 24 performs various kinds of image processing on an image obtained by imaging the intestinal wall 32 with the endoscope 16.

The display device 18 displays various kinds of information including images. An example of the display device 18 is a liquid crystal display or an EL display. A tablet terminal with a display may be used instead of or together with the display device 18.

The display device 18 displays a screen 35. The screen 35 includes a plurality of display regions. The plurality of display regions are arranged side by side on the screen 35. In the example illustrated in FIG. 1, a first display region 36 and a second display region 38 are depicted as an example of the plurality of display regions. The size of the first display region 36 is larger than the size of the second display region 38. The first display region 36 is used as a main display region, and the second display region 38 is used as a sub-display region. The relationship in size between the first display region 36 and the second display region 38 is not limited to this, and the first display region 36 and the second display region 38 may have any relationship in size so as to fit in the screen 35.

In the present embodiment, the screen 35 is an example of the “screen” according to the technology of the present disclosure, the second display region 38 is an example of the “second screen” according to the technology of the present disclosure, and the first display region 36 is an example of the “first screen” according to the technology of the present disclosure.

The first display region 36 displays an endoscopic moving image 39. The endoscopic moving image 39 is a moving image acquired by imaging the intestinal wall 32 with the endoscope 16 in the large intestine 28 of the subject 26. In the example illustrated in FIG. 1, a moving image in which the intestinal wall 32 appears is depicted as an example of the endoscopic moving image 39.

The intestinal wall 32 appearing in the endoscopic moving image 39 includes a lesion 42 (for example, in the example illustrated in FIG. 1, one lesion 42) as a region of interest (that is, an observation target region) to be gazed at by the doctor 12, and the doctor 12 can visually recognize the state of the intestinal wall 32 including the lesion 42 through the endoscopic moving image 39. In the present embodiment, the lesion 42 is an example of the “observation target region” and the “lesion” according to the technology of the present disclosure. The intestinal wall 32 including the lesion 42 is an example of the “imaging target region” according to the technology of the present disclosure.

There are various types of lesions 42, and the types of lesions 42 include, for example, neoplastic polyps and non-neoplastic polyps. The types of the neoplastic polyps include, for example, adenomatous polyps (for example, SSL). The types of the non-neoplastic polyps include, for example, hamartoma polyps, hyperplastic polyps, and inflammatory polyps. The types exemplified here are types considered in advance to be possible types of the lesion 42 when an endoscopic examination is performed on the large intestine 28, and the type of lesion differs depending on the organ on which the endoscopic examination is performed.

While the present embodiment provides an example embodiment in which one lesion 42 appears in the endoscopic moving image 39 for convenience of description, the technology of the present disclosure is not limited to this, and the technology of the present disclosure is also applicable in a case where a plurality of lesions 42 appear in the endoscopic moving image 39.

While the present embodiment exemplifies the lesion 42, this is merely an example. The region of interest (that is, the observation target region) to be gazed at by the doctor 12 may be an organ (for example, the duodenal papilla), a marked region, an artificial treatment tool (for example, an artificial clip), a treated region (for example, a region with a trace of removal of a polyp or the like), or the like.

The image displayed in the first display region 36 is one frame 40 included in a moving image configured to include a plurality of frames 40 along a time series. That is, the first display region 36 displays the plurality of frames 40 along a time series at a predetermined frame rate (for example, several tens of frames/second). In the present embodiment, the frame 40 is an example of the “medical image” and the “endoscopic image” according to the technology of the present disclosure.

An example of the moving image to be displayed in the first display region 36 is a live-view moving image. The live-view moving image is merely an example, and a moving image that is temporarily stored in a memory or the like before being displayed, like a post-view moving image, may be used. Alternatively, each frame included in a recording moving image stored in the memory or the like may be reproduced and displayed on the screen 35 (for example, the first display region 36) as the endoscopic moving image 39.

On the screen 35, the second display region 38 is adjacent to the first display region 36 and is displayed in a lower right portion of the screen 35 when viewed from the front. The second display region 38 may be displayed at any position within the screen 35 of the display device 18, but is preferably displayed at a position that enables comparison with the endoscopic moving image 39.

The second display region 38 displays medical information 44, which is information related to medical treatment. Examples of the medical information 44 include information and the like for assisting the doctor 12 in making a medical determination or the like. An example of the information and the like for assisting the doctor 12 in making a medical determination or the like is various kinds of information related to the subject 26 into which the endoscope 16 is inserted, various kinds of information obtained by performing processing using AI on the endoscopic moving image 39, and/or the like. The medical information 44 will be described in further detail below.

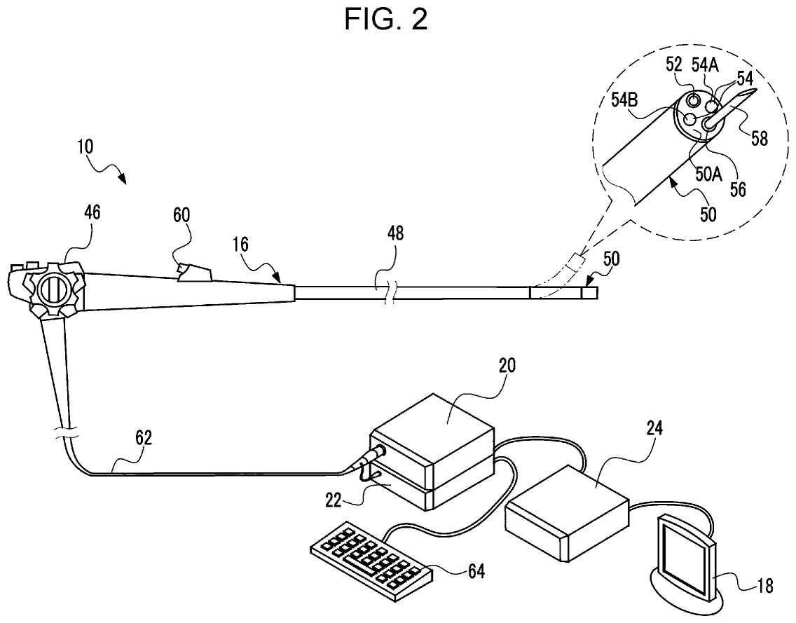

As an example, as illustrated in FIG. 2, the endoscope 16 includes an operation section 46 and an insertion section 48. The insertion section 48 partially bends in response to the operation section 46 being operated. When the doctor 12 (see FIG. 1) operates the operation section 46, the insertion section 48 is inserted into the large intestine 28 (see FIG. 1) while bending according to the shape of the large intestine 28.

The insertion section 48 has a tip portion 50 provided with a camera 52, an illumination device 54, and a treatment tool opening 56. The camera 52 and the illumination device 54 are provided on a tip surface 50A of the tip portion 50. While an example embodiment in which the camera 52 and the illumination device 54 are provided on the tip surface 50A of the tip portion 50 is given here, this is merely an example. The camera 52 and the illumination device 54 may be provided on a side surface of the tip portion 50 such that the endoscope 16 is configured as a side view endoscope.

The camera 52 is inserted into the body cavity of the subject 26 to perform imaging of an observation target region. In the present embodiment, the camera 52 performs imaging of the inside of the body of the subject 26 (for example, the inside of the large intestine 28) to acquire the endoscopic moving image 39. An example of the camera 52 is a CMOS camera. However, this is merely an example, and the camera 52 may be any other type of camera such as a CCD camera.

The illumination device 54 has illumination windows 54A and 54B. The illumination device 54 emits the light 30 (see FIG. 1) through the illumination windows 54A and 54B. Examples of the type of the light 30 to be emitted from the illumination device 54 include visible light (for example, white light or the like) and invisible light (for example, near-infrared light or the like). The illumination device 54 further emits special light through the illumination windows 54A and 54B. Examples of the special light include light for BLI and/or light for LCI. The camera 52 performs imaging of the inside of the large intestine 28 using an optical method, with the inside of the large intestine 28 irradiated with the light 30 from the illumination device 54.

The treatment tool opening 56 is an opening for allowing a treatment tool 58 to protrude from the tip portion 50. The treatment tool opening 56 is also used as a suction port for sucking blood, bodily waste, and the like, and as a delivery port for delivering a fluid.

The operation section 46 has a treatment tool insertion port 60 formed therein, and the treatment tool 58 is inserted into the insertion section 48 through the treatment tool insertion port 60. The treatment tool 58 passes through the insertion section 48 and protrudes to the outside from the treatment tool opening 56. In the example illustrated in FIG. 2, a puncture needle is illustrated as the treatment tool 58 protruding from the treatment tool opening 56. While a puncture needle is exemplified as the treatment tool 58, this is merely an example, and the treatment tool 58 may be gripping forceps, a papillotomy knife, a snare, a catheter, a guide wire, a cannula, a puncture needle with a guide sheath, and/or the like.

The endoscope 16 is connected to the light source device 20 and the control device 22 through a universal cord 62. The control device 22 is connected to the medical support device 24 and a reception device 64. The medical support device 24 is also connected to the display device 18. That is, the control device 22 is connected to the display device 18 through the medical support device 24.

Since the medical support device 24 is exemplified here as an external device for extending the functions implemented by the control device 22, an example embodiment in which the control device 22 and the display device 18 are indirectly connected through the medical support device 24 is given here, although this is merely an example. For example, the display device 18 may be directly connected to the control device 22. In this case, for example, the control device 22 may be mounted with the functions of the medical support device 24, or the control device 22 may be mounted with a function of causing a server (not illustrated) to execute the same process as a process (for example, a medical support process described below) executed by the medical support device 24 and receiving and using a process result obtained by the server.

The reception device 64 receives an instruction from the doctor 12 and outputs the received instruction to the control device 22 as an electrical signal. An example of the reception device 64 is a keyboard, a mouse, a touch panel, a foot switch, a microphone, a remote operation device, and/or the like.

The control device 22 controls the light source device 20, transmits and receives various signals to and from the camera 52, and transmits and receives various signals to and from the medical support device 24.

The light source device 20 emits light under the control of the control device 22 and supplies the light to the illumination device 54. The illumination device 54 incorporates a light guide, and the light supplied from the light source device 20 is emitted from the illumination windows 54A and 54B through the light guide. The control device 22 causes the camera 52 to perform imaging, acquires the endoscopic moving image 39 (see FIG. 1) from the camera 52, and outputs the endoscopic moving image 39 to a predetermined output destination (for example, the medical support device 24).

The medical support device 24 performs various kinds of image processing on the endoscopic moving image 39 input from the control device 22 to support medical treatment (here, as an example, endoscopy). The medical support device 24 outputs the endoscopic moving image 39 on which the various kinds of image processing have been performed to a predetermined output destination (for example, the display device 18).

While an example embodiment has been described in which the endoscopic moving image 39 output from the control device 22 is output to the display device 18 through the medical support device 24, this is merely an example. For example, in another aspect, the control device 22 and the display device 18 may be connected to each other, and the endoscopic moving image 39 on which image processing has been performed by the medical support device 24 may be displayed on the display device 18 through the control device 22.

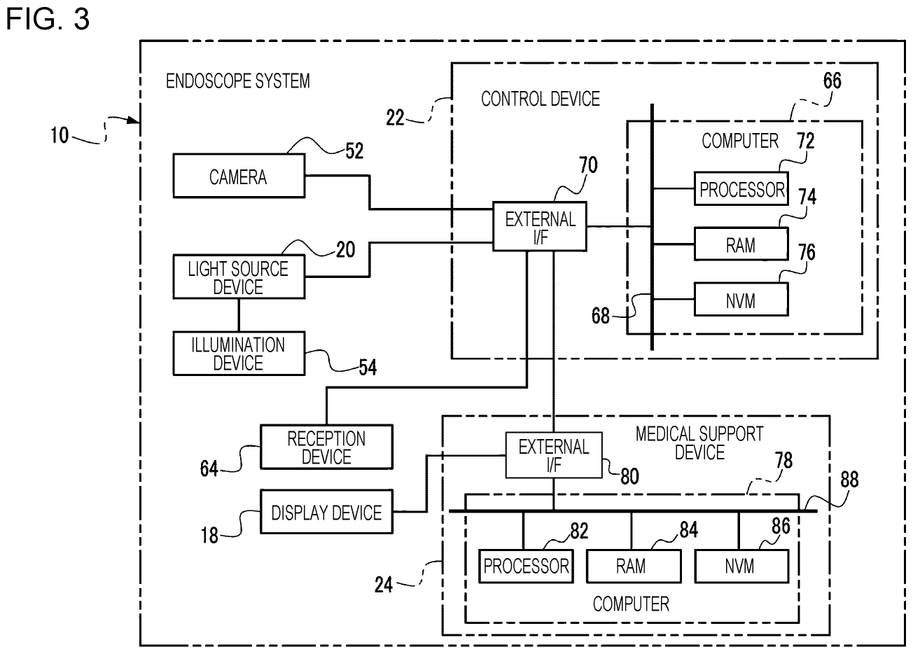

As an example, as illustrated in FIG. 3, the control device 22 includes a computer 66, a bus 68, and an external I/F 70. The computer 66 includes a processor 72, a RAM 74, and an NVM 76. The processor 72, the RAM 74, the NVM 76, and the external I/F 70 are connected to the bus 68.

For example, the processor 72 has at least one CPU and at least one GPU and controls the entire control device 22. The GPU operates under the control of the CPU and is responsible for performing various kinds of graphics-based processing, arithmetic operations using a neural network, and the like. The processor 72 may include one or more CPUs with integrated GPU functions, or may include one or more CPUs without integrated GPU functions. In the example illustrated in FIG. 3, the computer 66 is mounted with one processor 72. However, this is merely an example, and the computer 66 may be mounted with a plurality of processors 72.

The RAM 74 is a memory that temporarily stores information and is used as a work memory by the processor 72. The NVM 76 is a non-volatile storage device that stores various programs, various parameters, and the like. An example of the NVM 76 is a flash memory (for example, an EEPROM and/or an SSD). The flash memory is merely an example, and the NVM 76 may be any other non-volatile storage device such as an HDD, or a combination of two or more types of non-volatile storage devices.

The external I/F 70 handles transmission and reception of various kinds of information between the processor 72 and one or more devices (hereinafter also referred to as “first external devices”) external to the control device 22. An example of the external I/F 70 is a USB interface.

The camera 52 is connected to the external I/F 70 as one of the first external devices, and the external I/F 70 handles transmission and reception of various kinds of information between the camera 52 and the processor 72. The processor 72 controls the camera 52 through the external I/F 70. Further, the processor 72 acquires the endoscopic moving image 39 (see FIG. 1), which is obtained by imaging the inside of the large intestine 28 (see FIG. 1) using the camera 52, through the external I/F 70.

The light source device 20 is connected to the external I/F 70 as one of the first external devices, and the external I/F 70 handles transmission and reception of various kinds of information between the light source device 20 and the processor 72. The light source device 20 supplies light to the illumination device 54 under the control of the processor 72. The illumination device 54 emits the light supplied from the light source device 20.

The reception device 64 is connected to the external I/F 70 as one of the first external devices, and the processor 72 acquires an instruction received by the reception device 64 through the external I/F 70 and executes a process corresponding to the acquired instruction.

The medical support device 24 includes a computer 78 and an external I/F 80. The computer 78 includes a processor 82, a RAM 84, and an NVM 86. The processor 82, the RAM 84, the NVM 86, and the external I/F 80 are connected to a bus 88. In the present embodiment, the medical support device 24 is an example of the “medical support device” according to the technology of the present disclosure, the computer 78 is an example of the “computer” according to the technology of the present disclosure, and the processor 82 is an example of the “processor” according to the technology of the present disclosure.

Since the hardware configuration (that is, the processor 82, the RAM 84, and the NVM 86) of the computer 78 is basically the same as the hardware configuration of the computer 66, the description of the hardware configuration of the computer 78 will be omitted here.

The external I/F 80 handles transmission and reception of various kinds of information between the processor 82 and one or more devices (hereinafter also referred to as “second external devices”) external to the medical support device 24. An example of the external I/F 80 is a USB interface.

The control device 22 is connected to the external I/F 80 as one of the second external devices. In the example illustrated in FIG. 3, the external I/F 70 of the control device 22 is connected to the external I/F 80. The external I/F 80 handles transmission and reception of various kinds of information between the processor 82 of the medical support device 24 and the processor 72 of the control device 22. For example, the processor 82 acquires the endoscopic moving image 39 (see FIG. 1) from the processor 72 of the control device 22 through the external I/Fs 70 and 80, and performs various kinds of image processing on the acquired endoscopic moving image 39.

The display device 18 is connected to the external I/F 80 as one of the second external devices. The processor 82 controls the display device 18 through the external I/F 80 to display various kinds of information (for example, the endoscopic moving image 39 and the like on which the various kinds of image processing have been performed) on the display device 18.

In an endoscopic examination, the doctor 12 determines whether the lesion 42 appearing in the endoscopic moving image 39 requires medical treatment, while checking the endoscopic moving image 39 through the display device 18, and performs medical treatment on the lesion 42, if necessary. The size of the lesion 42 is a determination factor important for determining whether medical treatment is necessary.

The recent development of machine learning has enabled the AI-based detection and classification of the lesion 42 based on the endoscopic moving image 39. Application of this technique makes it possible to measure the size of the lesion 42 from the endoscopic moving image 39. Accurately measuring the size of the lesion 42 and presenting the measurement result to the doctor 12 is very useful for the doctor 12 to perform medical treatment on the lesion 42. For example, when the lesion 42 is a colonic polyp, the larger the size of the colonic polyp, the more possible it is for the colonic polyp to become cancerous or the more possible it is for the colonic polyp to develop into cancer. When the size of the colonic polyp is greater than or equal to a reference value, the doctor 12 decides to perform a medical treatment (for example, excision) on the colonic polyp. An example of the reference value for the size of the colonic polyp is 5 mm, 10 mm, or the like.

However, if the size of the colonic polyp is slightly larger or slightly smaller than the reference value, the doctor 12 is expected to hesitate to decide whether to perform a medical treatment on the colonic polyp or to observe the colonic polyp without performing any medical treatment.

Depending on the appearance of the lesion 42 in the endoscopic moving image 39 (for example, the appearance of the lesion 42 in the endoscopic moving image 39 in a case where the relative positional relationship between the lesion 42 and the camera 52 is not a positional relationship expected in advance), a size less than the reference value may be presented to the doctor 12 although the actual size of the lesion 42 is greater than or equal to the reference value. Depending on the appearance of the lesion 42 in the endoscopic moving image 39, conversely, a size greater than or equal to the reference value may be presented to the doctor 12 although the actual size of the lesion 42 is less than the reference value. If such an erroneously measured size is presented to the doctor 12, the doctor 12 may make a wrong clinical decision. Thus, it is very important to prevent such a situation from occurring.

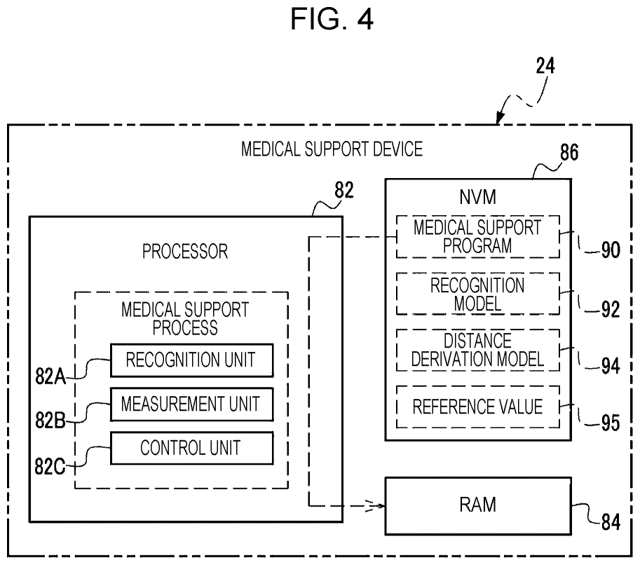

In view of such circumstances, in the present embodiment, as an example, as illustrated in FIG. 4, the processor 82 of the medical support device 24 performs a medical support process.

The NVM 86 stores a medical support program 90. The medical support program 90 is an example of the “program” according to the technology of the present disclosure. The processor 82 reads the medical support program 90 from the NVM 86 and executes the read medical support program 90 on the RAM 84 to perform the medical support process. The medical support process is implemented by the processor 82 operating as a recognition unit 82A, a measurement unit 82B, and a control unit 82C in accordance with the medical support program 90 executed on the RAM 84.

The NVM 86 stores a recognition model 92, a distance derivation model 94, and a reference value 95. As described in detail below, the recognition model 92 is used by the recognition unit 82A, the distance derivation model 94 is used by the measurement unit 82B, and the reference value 95 is used by the control unit 82C.

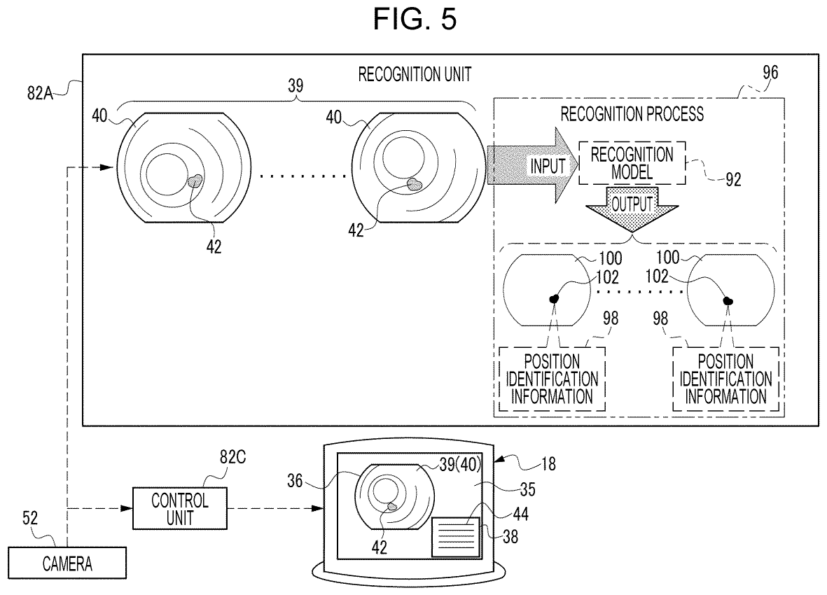

As an example, as illustrated in FIG. 5, the recognition unit 82A and the control unit 82C acquire each of a plurality of frames 40 along a time series included in the endoscopic moving image 39 generated by imaging with the camera 52 in accordance with an imaging frame rate (for example, several tens of frames/second) from the camera 52 frame by frame along a time series.

The control unit 82C outputs the endoscopic moving image 39 to the display device 18. For example, the control unit 82C displays the endoscopic moving image 39 in the first display region 36 as a live view image. That is, each time the control unit 82C acquires a frame 40 from the camera 52, the control unit 82C sequentially displays the acquired frame 40 in the first display region 36 in accordance with a display frame rate (for example, several tens of frames/second). The control unit 82C further displays the medical information 44 in the second display region 38. Further, for example, the control unit 82C updates the content (for example, the medical information 44) displayed in the second display region 38 in accordance with the content displayed in the first display region 36.

The recognition unit 82A uses the endoscopic moving image 39 acquired from the camera 52 to recognize the lesion 42 in the endoscopic moving image 39. That is, the recognition unit 82A sequentially performs a recognition process 96 on each of the plurality of frames 40 along a time series included in the endoscopic moving image 39 acquired from the camera 52 to recognize the lesion 42 appearing in each of the frames 40. For example, the recognition unit 82A recognizes the geometric characteristics (for example, the position, the shape, and the like) of the lesion 42, the type of the lesion 42, the category of the lesion 42 (for example, pedunculated, sub-pedunculated, sessile, superficial elevated, superficial flat, superficial depressed, and the like), and the like.

Each time a frame 40 is acquired, the recognition unit 82A performs the recognition process 96 on the acquired frame 40. The recognition process 96 is a process for recognizing the lesion 42 by a method using AI. In the present embodiment, for example, an AI-based object recognition process using a segmentation method (for example, semantic segmentation, instance segmentation, and/or panoptic segmentation) is used as the recognition process 96.

A process using the recognition model 92 is performed as the recognition process 96. The recognition model 92 is a trained model for object recognition using an AI-based segmentation method. An example of the trained model for object recognition using an AI-based segmentation method is a model for semantic segmentation. An example of the model for semantic segmentation is an encoder-decoder structure model. An example of the encoder-decoder structure model is a U-Net model, an HRNet model, or the like. In the present embodiment, the recognition process 96 is an example of the “object recognition process” according to the technology of the present disclosure.

The recognition model 92 is optimized by training a neural network through machine learning using first training data. The first training data is a dataset including a plurality of pieces of data (that is, data for a plurality of frames) in which first example data and first ground-truth data are associated with each other.

The first example data is an image corresponding to the frame 40. The first ground-truth data is ground-truth data (that is, an annotation) for the first example data. An example of the first ground-truth data is an annotation for identifying the geometric characteristics, type, and category of a lesion appearing in an image used as the first example data.

The recognition unit 82A acquires a frame 40 from the camera 52 and inputs the acquired frame 40 to the recognition model 92. Accordingly, each time a frame 40 is input, the recognition model 92 identifies the geometric characteristics of the lesion 42 appearing in the input frame 40 and outputs information that can identify the geometric characteristics. In the example illustrated in FIG. 5, position identification information 98 that can identify the position of the lesion 42 in the frame 40 is depicted as an example of the information that can identify the geometric characteristics. Further, the recognition unit 82A acquires, from the recognition model 92, information indicating the type and category of the lesion 42 appearing in the frame 40 input to the recognition model 92.

Each time a frame 40 is input to the recognition model 92, the recognition unit 82A acquires, from the recognition model 92, a probability map 100 related to the frame 40 input to the recognition model 92. The probability map 100 is a map in which the distribution of the position of the lesion 42 in the frame 40 is expressed in terms of a probability, which is an example of a measure of the likelihood. The probability map 100 is typically referred to also as a reliability map, a certainty map, or the like.

The probability map 100 includes a segmentation image 102 that defines the lesion 42 recognized by the recognition unit 82A. The segmentation image 102 is an image region for identifying the position of the lesion 42, which is recognized by performing the recognition process 96 on the frame 40, in the frame 40 (that is, an image displayed in a display style that can identify the position where the lesion 42 is most likely to be present in the frame 40). The segmentation image 102 is associated with the position identification information 98 by the recognition unit 82A. Examples of the position identification information 98 in this case include coordinates for identifying the position of the segmentation image 102 in the frame 40.

The probability map 100 may be displayed on the screen 35 (for example, in the second display region 38) as the medical information 44 by the control unit 82C. In this case, the probability map 100 displayed on the screen 35 is updated in accordance with the display frame rate applied to the first display region 36. That is, the display of the probability map 100 in the second display region 38 (that is, the display of the segmentation image 102) is updated in synchronization with the display timing of the endoscopic moving image 39 displayed in the first display region 36. This configuration allows the doctor 12 to grasp the schematic position of the lesion 42 in the endoscopic moving image 39 displayed in the first display region 36 by referring to the probability map 100 displayed in the second display region 38 while observing the endoscopic moving image 39 displayed in the first display region 36.

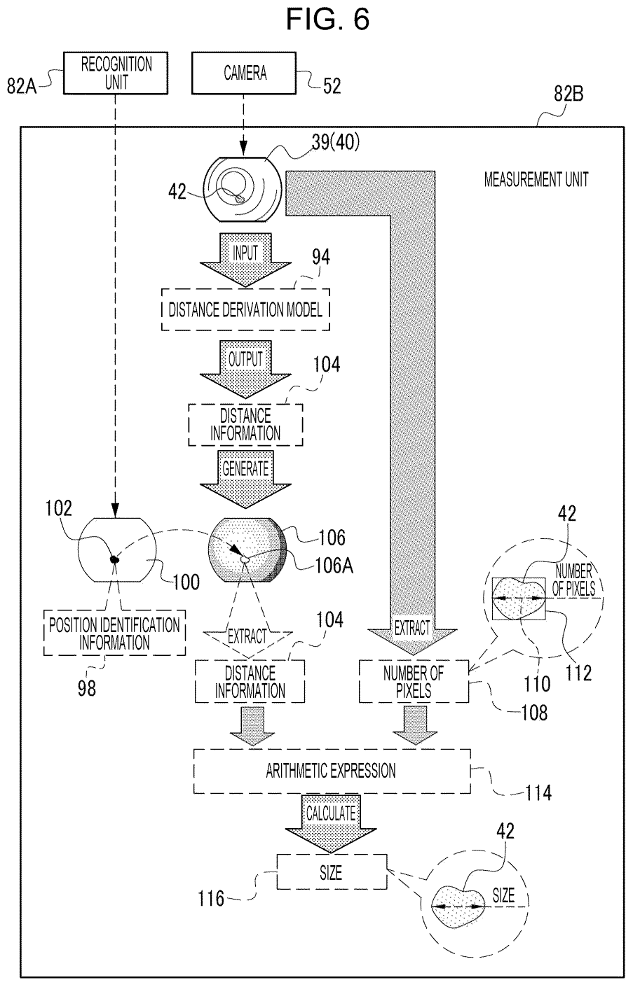

As an example, as illustrated in FIG. 6, the measurement unit 82B acquires a frame 40 from the camera 52 and acquires a size 116 of the lesion 42 appearing in the frame 40 acquired from the camera 52 (as an example, the frame 40 used in the recognition process 96). The size 116 of the lesion 42 appearing in the frame 40 is acquired by measuring the size 116 with the measurement unit 82B. The measurement unit 82B measures the size 116, based on the frame 40. In the present embodiment, the measurement unit 82B measures the size 116 of the lesion 42 in time series, based on each of the plurality of frames 40 included in the endoscopic moving image 39 acquired from the camera 52. The size 116 of the lesion 42 refers to the size of the lesion 42 in real space. In the following, the size of the lesion 42 in real space is also referred to as an “actual size”, for convenience of description.

To implement the measurement of the size 116 of the lesion 42, the measurement unit 82B acquires distance information 104 of the lesion 42, based on the frames 40 acquired from the camera 52. The distance information 104 is information indicating the distance from the camera 52 (that is, the observation position) to the intestinal wall 32 (see FIG. 1) including the lesion 42. While the distance from the camera 52 to the intestinal wall 32 including the lesion 42 is exemplified here, this is merely an example. Instead of the distance, a numerical value indicating the depth from the camera 52 to the intestinal wall 32 including the lesion 42 (for example, a plurality of numerical values defining depths in a stepwise manner (for example, numerical values in several steps to several tens of steps)) may be used.

The distance information 104 is acquired for each of all the pixels constituting the frame 40. The distance information 104 may be acquired for each block (for example, a pixel group constituted by several pixels to several hundreds of pixels), which is larger than a pixel in the frame 40.

The measurement unit 82B acquires the distance information 104 by, for example, deriving the distance information 104 by using an AI-based method. In the present embodiment, the distance derivation model 94 is used to derive the distance information 104.

The distance derivation model 94 is optimized by training the neural network through machine learning using second training data. The second training data is a dataset including a plurality of pieces of data (that is, data for a plurality of frames) in which second example data and second ground-truth data are associated with each other.

The second example data is an image corresponding to the frame 40. The second ground-truth data is ground-truth data (that is, an annotation) for the second example data. An example of the second ground-truth data is an annotation for identifying a distance corresponding to each pixel appearing in an image used as the second example data.

The measurement unit 82B acquires the frame 40 from the camera 52 and inputs the acquired frame 40 to the distance derivation model 94. As a result, the distance derivation model 94 outputs the distance information 104 on a pixel-by-pixel basis in the frame 40 that has been input. That is, in the measurement unit 82B, information indicating the distance from the position of the camera 52 (for example, the position of the image sensor, the objective lens, or the like mounted in the camera 52) to the intestinal wall 32 appearing in the frame 40 is output from the distance derivation model 94 as the distance information 104 on a pixel-by-pixel basis in the frame 40.

The measurement unit 82B generates a distance image 106, based on the distance information 104 output from the distance derivation model 94. The distance image 106 is an image in which the distance information 104 is distributed in units of pixels included in the endoscopic moving image 39.

The measurement unit 82B acquires the position identification information 98 assigned to the segmentation image 102 on the probability map 100 obtained by the recognition unit 82A. The measurement unit 82B refers to the position identification information 98 and extracts the distance information 104 from a segmentation-corresponding region 106A in the distance image 106. The segmentation-corresponding region 106A is a region corresponding to a position identified from the position identification information 98 in the distance image 106. The distance information 104 extracted from the segmentation-corresponding region 106A includes, for example, the distance information 104 corresponding to the position (for example, centroid) of the lesion 42, or a statistical measure (for example, the median, mean, or mode) based on the distance information 104 for a plurality of pixels (for example, all the pixels) included in the lesion 42.

The measurement unit 82B extracts the number of pixels 108 from the frame 40. The number of pixels 108 is the number of pixels on a line segment 110 crossing an image region located at a position identified from the position identification information 98 (that is, an image region indicating the lesion 42) within an entire image region of the frame 40 input to the distance derivation model 94. An example of the line segment 110 is the longest line segment parallel to the long sides of a rectangular frame 112 circumscribing the image region indicating the lesion 42. The line segment 110 is merely an example. Instead of the line segment 110, the longest line segment parallel to the short sides of the rectangular frame 112 circumscribing the image region indicating the lesion 42 may be used.

The measurement unit 82B calculates the size 116 of the lesion 42, based on the distance information 104 extracted from the segmentation-corresponding region 106A in the distance image 106 and the number of pixels 108 extracted from the frame 40. The size 116 is calculated using an arithmetic expression 114. The arithmetic expression 114 is an arithmetic expression in which the distance information 104 and the number of pixels 108 are independent variables and the size 116 is a dependent variable. The measurement unit 82B inputs the distance information 104 extracted from the distance image 106 and the number of pixels 108 extracted from the frame 40 to the arithmetic expression 114. The arithmetic expression 114 outputs the size 116 corresponding to the distance information 104 and the number of pixels 108 that have been input. In the present embodiment, the size 116 is an example of the “size” and the “size of the observation target region in at least one direction” according to the technology of the present disclosure.

While the length of the lesion 42 in real space is exemplified as the size 116, the technology of the present disclosure is not limited to this. The size 116 may be the surface area or volume of the lesion 42 in real space. In this case, for example, as the arithmetic expression 114, an arithmetic expression is used in which the number of pixels in the entire image region indicating the lesion 42 and the distance information 104 are independent variables and the surface area or volume of the lesion 42 in real space is a dependent variable.

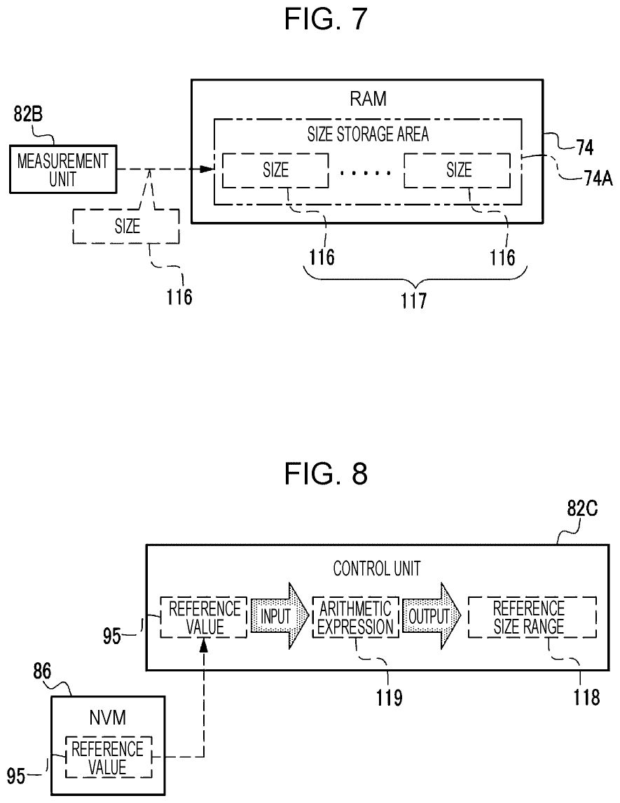

As an example, as illustrated in FIG. 7, the RAM 74 is provided with a size storage area 74A, and the measurement unit 82B stores the measured size 116 in the size storage area 74A as a previous size 117. Each time the measurement unit 82B measures the size 116 along a time series, the measured size 116 is stored in the size storage area 74A as a previous size 117 in a FIFO manner. For example, the size storage area 74A stores, in time series, previous sizes 117 of the lesion 42 appearing in a plurality of frames 40 along a time series (for example, a plurality of frames 40 determined within a range of about several to several hundreds of frames 40).

As an example, as illustrated in FIG. 8, the control unit 82C acquires the reference value 95 from the NVM 86. The reference value 95 is a reference value for clinical decision making. The reference value 95 is determined based on medical knowledge. An example of the clinical decision making is decision making on whether to excise the lesion 42 from the intestinal wall 32. In the present embodiment, for example, the lesion 42 is a colonic polyp, and the reference value 95 for excising the colonic polyp from the intestinal wall 32 is 5.0 mm. While a colonic polyp is given as an example of the lesion 42, the lesion 42 may be a lesion other than a colonic polyp so long as the reference value 95 is determined in accordance with the lesion. The reference value 95 may be a fixed value or a variable value that is changed in accordance with an instruction received by the reception device 64 and/or various conditions.

The control unit 82C determines a reference size range 118, based on the reference value 95. The reference size range 118 is a size range determined with respect to the reference value 95 and is used for comparison with the size 116. For example, the reference size range 118 is a size range that may make the doctor 12 hesitate to make a clinical decision by referring to the size 116. When the reference value 95 is 5.0 mm, an example of the reference size range 118 is a range greater than or equal to 4.0 mm and less than or equal to 6.0 mm.

The reference size range 118 is determined using an arithmetic expression 119. The arithmetic expression 119 is an arithmetic expression in which the reference value 95 is an independent variable and the reference size range 118 is a dependent variable. The control unit 82C inputs the reference value 95 acquired from the NVM 86 to the arithmetic expression 119. The arithmetic expression 119 outputs the reference size range 118 corresponding to the reference value 95 that has been input. In the present embodiment, the reference size range 118 is an example of the “size range” according to the technology of the present disclosure.

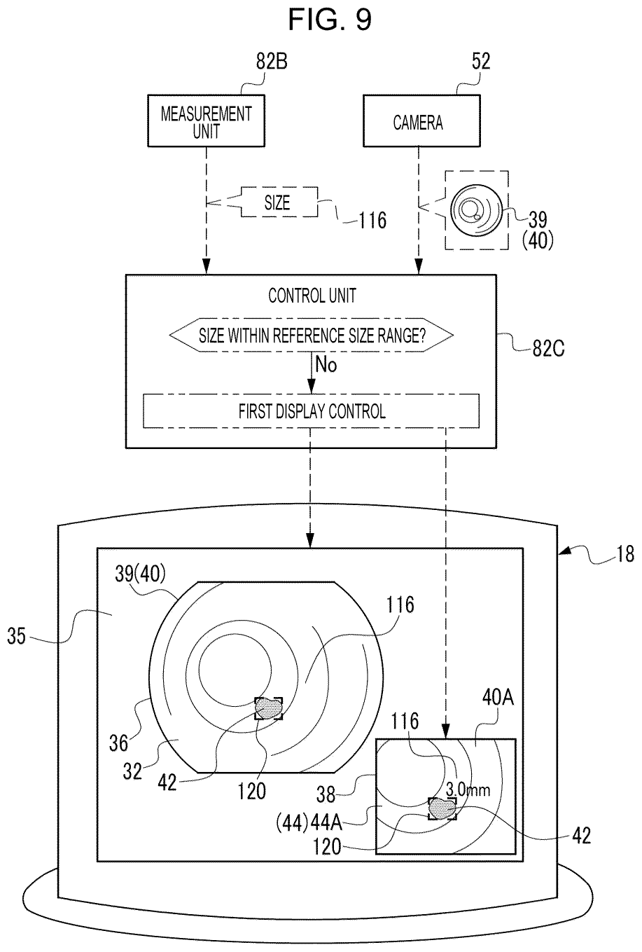

As an example, as illustrated in FIG. 9, the control unit 82C acquires the size 116 from the measurement unit 82B. The control unit 82C further acquires, from the camera 52, the frame 40 used for the measurement of the size 116 by the measurement unit 82B.

The control unit 82C determines whether the size 116 acquired from the measurement unit 82B falls within the reference size range 118. If the size 116 acquired from the measurement unit 82B falls outside the reference size range 118, the control unit 82C performs first display control on the display device 18.

When the control unit 82C performs the first display control, the frame 40 acquired from the camera 52 is displayed in the first display region 36, and a lesion position identification mark 120 is displayed in the frame 40. The lesion position identification mark 120 is a mark that can identify the position of the lesion 42 appearing in the frame 40 (in other words, a mark that can identify the position of the lesion 42 in the frame 40). In the example illustrated in FIG. 9, as an example of the lesion position identification mark 120, four L-shaped marks obtained by removing the four corners of the rectangular frame 112 (see FIG. 6) are illustrated. This is merely an example, and the contour of the image region indicating the lesion 42 appearing in the frame 40 may be highlighted, or the rectangular frame 112 may be displayed. In a case where the recognition process 96 is an AI-based process using a bounding box method, a bounding box corresponding to the lesion 42 appearing in the frame 40 may be displayed in the frame 40 as the lesion position identification mark 120.

For example, the lesion position identification mark 120 is displayed superimposed on the frame 40. The superimposed display of the lesion position identification mark 120 is merely an example, and the lesion position identification mark 120 may be displayed embedded in the frame 40. When the lesion position identification mark 120 is to be displayed superimposed on the frame 40, the lesion position identification mark 120 may be displayed superimposed on the frame 40 by using an alpha blending method. In the present embodiment, the lesion position identification mark 120 is an example of the “position information” according to the technology of the present disclosure.

When the control unit 82C performs the first display control, furthermore, a size-attached local image 44A is displayed in the second display region 38 as the medical information 44. The size-attached local image 44A has a local image 40A. The local image 40A is an image obtained by extracting a local portion of the frame 40 displayed in the first display region 36. The local image 40A depicts the lesion 42 having the size 116 to be measured by the measurement unit 82B. In the present embodiment, the local image 40A is an example of the “image indicating the observation target region” according to the technology of the present disclosure.

Like the frame 40 displayed in the first display region 36, the local image 40A displays the lesion position identification mark 120. The local image 40A further displays the size 116 acquired from the measurement unit 82B.

For example, the size 116 is displayed superimposed on the local image 40A. The superimposed display of the size 116 is merely an example, and the size 116 may be displayed embedded in the local image 40A. When the size 116 is to be displayed superimposed on the local image 40A, the size 116 may be displayed superimposed on the local image 40A by using an alpha blending method.

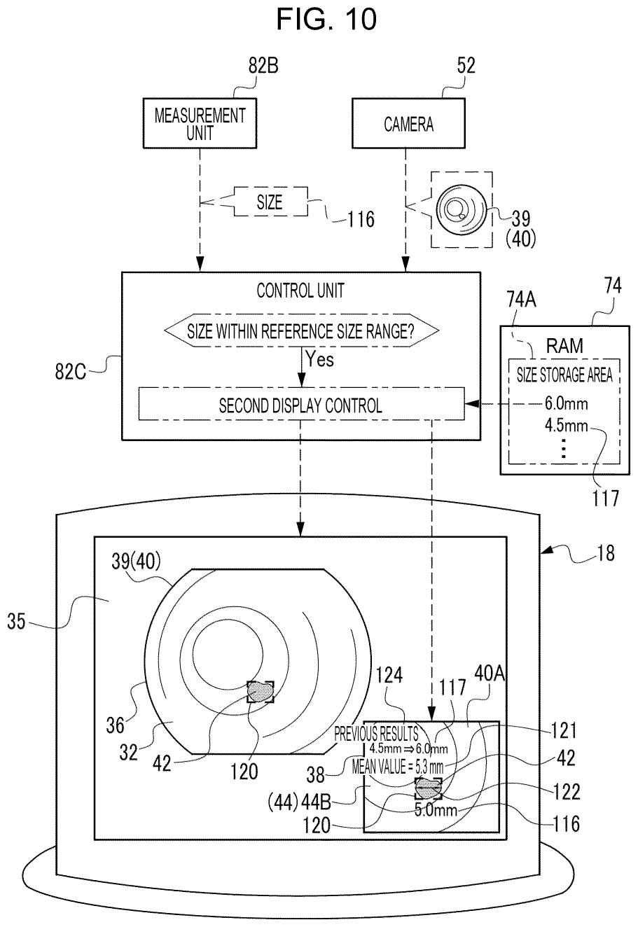

As an example, as illustrated in FIG. 10, if the size 116 acquired from the measurement unit 82B by the control unit 82C falls within the reference size range 118, the control unit 82C performs second display control on the display device 18. When the size 116 falls within the reference size range 118, this means that the size 116 is a size that may make the doctor 12 hesitate to make a clinical decision.

In the present embodiment, accordingly, the control unit 82C performs the second display control to display an image similar to that in the example illustrated in FIG. 9 in the first display region 36 and display auxiliary information 44B in the second display region 38 as the medical information 44. The auxiliary information 44B is information for assisting in clinical decision making (for example, assisting the doctor 12 in making a clinical decision).

The auxiliary information 44B also has the local image 40A, like the size-attached local image 44A. The local image 40A displays previous results 124. The previous results 124 include a plurality of latest previous sizes 117 (for example, the previous sizes 117 in the latest two frames) among the plurality of previous sizes 117 along the time series stored in the size storage area 74A. The plurality of latest previous sizes 117 included in the previous results 124 displayed in the second display region 38 are information enabling the identification of a range of variation in the size 116 identified when the size 116 is measured by the measurement unit 82B based on the plurality of frames 40. In the present embodiment, the plurality of latest previous sizes 117 included in the previous results 124 are an example of the “size variation range information” according to the technology of the present disclosure.

While an example embodiment in which the previous results 124 include a plurality of latest previous sizes 117 is given here, this is merely an example, and the previous results 124 may include a plurality of statistical sizes. The term “statistical size” refers to a statistical measure (for example, the mean, median, deviation, standard deviation, mode, maximum, minimum, and/or the like) based on a plurality of previous sizes 117 obtained at intervals of a plurality of frames. The plurality of latest previous sizes 117 included in the previous results 124 may be represented by a graph (for example, a line graph, a bar graph, and/or the like) and/or a table (for example, a matrix table or the like). The graph and/or the table may have any content that enables the identification of changes over time in the plurality of previous sizes 117 stored in the size storage area 74A. The content of the graph and/or the content of the table is updated in response to the update of the plurality of previous sizes 117 stored in the size storage area 74A. While an example embodiment in which the previous results 124 include a plurality of latest previous sizes 117 is given here, this is merely an example, and the previous results 124 may include any two or more previous sizes 117 along the time series stored in the size storage area 74A. Alternatively, the previous results 124 may include one previous size 117 stored in the size storage area 74A.

The previous results 124 further include a mean value 121. The mean value 121 is, for example, the mean value of the plurality of latest previous sizes 117 included in the previous results 124. The mean value 121 may be a mean value of a plurality of previous sizes 117 stored in the size storage area 74A (for example, all the previous sizes 117 or a plurality of previous sizes 117 in a plurality of most recent frames). While the mean value 121 is exemplified here, this is merely an example. Together with or instead of the mean value 121, a statistical measure such as a median, mode, deviation, standard deviation, maximum, and/or minimum may be used. In the present embodiment, the mean value 121 is an example of the “statistical measure” according to the technology of the present disclosure.

Further, the auxiliary information 44B displayed in the second display region 38 includes measurement direction information 122 that enables the identification of a measurement direction used to measure the size 116. A dimension line is used as an example of the measurement direction information 122. An example of the dimension line used as the measurement direction information 122 is a dimension line using the line segment 110 (see FIG. 6).

While an example embodiment in which a dimension line is used as an example of the measurement direction information 122 is given here, this is merely an example. Instead of the dimension line, text information enabling the identification of a measurement direction used to measure the size 116, an image other than the dimension line (for example, an arrow indicating the measurement direction), or the like may be used, and any information is applicable so long as the information enables the identification of a measurement direction used to measure the size 116.

Each time the measurement unit 82B measures the size 116, the control unit 82C displays the latest size 116 in the second display region 38. That is, the size 116 displayed in the second display region 38 is updated to the latest size 116 each time the measurement unit 82B measures the size 116. The latest size 116 may be displayed in the first display region 36. The previous results 124 may be displayed in the first display region 36, and the previous results 124 are updated in response to the size 116 being measured by the measurement unit 82B. The lesion position identification mark 120 may be displayed in the first display region 36 or the second display region 38. The lesion position identification mark 120 is updated each time the recognition process 96 is performed on the frame 40. The various kinds of information displayed on the screen 35 may be updated for each of the plurality of frames 40.

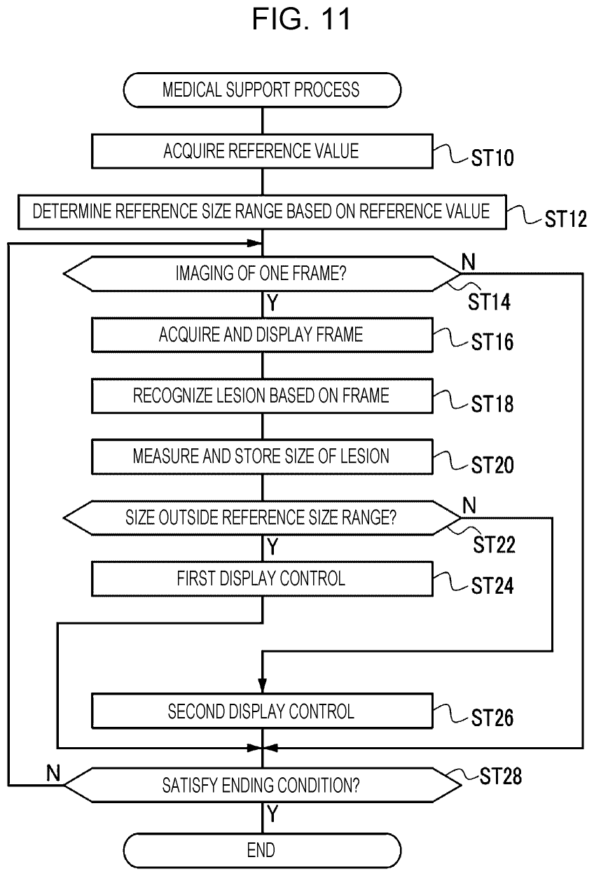

Next, the operation of a portion, according to the technology of the present disclosure, of the endoscope system 10 will be described with reference to FIG. 11. The flow of the medical support process illustrated in FIG. 11 is an example of the “medical support method” according to the technology of the present disclosure.

In the medical support process illustrated in FIG. 11, first, in step ST10, the control unit 82C acquires the reference value 95 from the NVM 86 (see FIG. 8). After the processing of step ST10 is performed, the medical support process proceeds to step ST12.

In step ST12, the control unit 82C determines the reference size range 118, based on the reference value 95 acquired from the NVM 86 in step ST10 (see FIG. 8). After the processing of step ST12 is performed, the medical support process proceeds to step ST14.

In step ST14, the recognition unit 82A determines whether imaging of one frame has been performed in the large intestine 28 by the camera 52. If imaging of one frame has not been performed in the large intestine 28 by the camera 52 in step ST14, the determination is negative, and the medical support process proceeds to step ST28. If imaging of one frame has been performed in the large intestine 28 by the camera 52 in step ST14, the determination is affirmative, and the medical support process proceeds to step ST16.

In step ST16, the recognition unit 82A and the control unit 82C acquire a frame 40 obtained by imaging the large intestine 28 with the camera 52. Then, the control unit 82C displays the frame 40 in the first display region 36 (see FIGS. 5, 9, and 10). For convenience of description, it is assumed here that the lesion 42 appears in the frame 40. After the processing of step ST16 is performed, the medical support process proceeds to step ST18.

In step ST18, the recognition unit 82A performs the recognition process 96 on the frame 40 acquired in step ST16 to recognize the lesion 42 appearing in the frame 40 (see FIG. 5). After the processing of step ST18 is performed, the medical support process proceeds to step ST20.

In step ST20, the measurement unit 82B measures the size 116 of the lesion 42 appearing in the frame 40, based on the frame 40 acquired in step ST16 and a recognition result obtained by the recognition process 96 performed in step ST18 (see FIG. 6). Then, the measurement unit 82B stores the measured size 116 in the size storage area 74A as a previous size 117 in a FIFO manner (see FIG. 7). After the processing of step ST20 is performed, the medical support process proceeds to step ST22.

In step ST22, the control unit 82C determines whether the size 116 measured in step ST20 falls outside the reference size range 118 determined in step ST12. If, in step ST22, the size 116 measured in step ST20 does not fall outside the reference size range 118 determined in step ST12, the determination is negative, and the medical support process proceeds to step ST26. If, in step ST22, the size 116 measured in step ST20 falls outside the reference size range 118 determined in step ST12, the determination is positive, and the medical support process proceeds to step ST24.

In step ST24, the control unit 82C performs the first display control on the display device 18 (see FIG. 9). As a result, the frame 40 acquired in step ST16 is displayed in the first display region 36, and the size-attached local image 44A is displayed in the second display region 38 (see FIG. 9). After the processing of step ST24 is performed, the medical support process proceeds to step ST28.

In step ST26, the control unit 82C performs the second display control on the display device 18 (see FIG. 10). As a result, the frame 40 acquired in step ST16 is displayed in the first display region 36, and the auxiliary information 44B is displayed in the second display region 38. After the processing of step ST26 is performed, the medical support process proceeds to step ST28.

In step ST28, the control unit 82C determines whether a condition for ending the medical support process is satisfied. An example of the condition for ending the medical support process is a condition in which an instruction to end the medical support process is given to the endoscope system 10 (for example, a condition in which the instruction to end the medical support process is received by the reception device 64).

If the condition for ending the medical support process is not satisfied in step ST28, the determination is negative, and the medical support process proceeds to step ST14. If the condition for ending the medical support process is satisfied in step ST28, the determination is affirmative, and the medical support process ends.

As described above, the reference size range 118 is, for example, a size range (for example, 4.0 mm to 6.0 mm) to be applied to colonic polyps, and the doctor 12 may hesitate to make a clinical decision on the lesion 42 when the size 116 falls within the reference size range 118. In the endoscope system 10, accordingly, when the size 116 acquired by the measurement unit 82B falls within the reference size range 118, the auxiliary information 44B is displayed in the second display region 38. The auxiliary information 44B displayed in the second display region 38 is information for assisting in clinical decision making for the lesion 42. Accordingly, the endoscope system 10 can contribute to improvement in the accuracy of clinical decision making for the lesion 42. For example, when the clinical decision making for the lesion 42 is clinical decision making as to whether to excise the lesion 42 from the intestinal wall 32, it is possible to contribute to improvement in the accuracy of clinical decision making as to whether to excise the lesion 42 from the intestinal wall 32.

In the endoscope system 10, furthermore, the measurement unit 82B measures the size 116, based on the frame 40. Accordingly, the endoscope system 10 enables accurate acquisition of the actual size of the lesion 42 without taking time and effort, as compared to the visual estimation of the actual size of the lesion 42.

The auxiliary information 44B displayed in the second display region 38 includes the local image 40A. The local image 40A is an image obtained by extracting a local portion of the frame 40 displayed in the first display region 36. The auxiliary information 44B displayed in the second display region 38 further includes the lesion position identification mark 120 as information enabling the identification of the position of the lesion 42 in the frame 40. The auxiliary information 44B displayed in the second display region 38 further includes the size 116 measured by the measurement unit 82B. The auxiliary information 44B further includes a plurality of latest previous sizes 117 as information enabling the identification of the range of variation in the size 116 identified when the size 116 is measured by the measurement unit 82B based on the plurality of frames 40. The auxiliary information 44B also includes the mean value 121. The mean value 121 is the mean value of the plurality of latest previous sizes 117 displayed in the second display region 38. The auxiliary information 44B includes the measurement direction information 122 that enables the identification of a measurement direction used to measure the size 116. As described above, the auxiliary information 44B displayed in the second display region 38 includes the local image 40A, the lesion position identification mark 120, the size 116, the plurality of latest previous sizes 117, the mean value 121, and the measurement direction information 122. This allows the doctor 12 to accurately make a clinical decision on the lesion 42 by referring to the auxiliary information 44B displayed in the second display region 38.

In the endoscope system 10, furthermore, the reference value 95 used to determine the reference size range 118 is determined based on medical knowledge. Accordingly, the endoscope system 10 enables the doctor 12 to make a clinical decision on the lesion 42 based on medical knowledge.

In the endoscope system 10, furthermore, the auxiliary information 44B is displayed in the second display region 38. This allows the doctor 12 to visually recognize the auxiliary information 44B.

In the endoscope system 10, furthermore, the frame 40 in which the lesion 42 appears is displayed in the first display region 36, and the auxiliary information 44B is displayed in the second display region 38 arranged in a manner that enables comparison with the first display region 36. Accordingly, the endoscope system 10 enables the doctor 12 to make a clinical decision on the lesion 42 while comparing the frame 40 and the auxiliary information 44B.

While the embodiment described above provides an example embodiment in which the reference value 95 is stored in the NVM 86 and the control unit 82C acquires the reference value 95 from the NVM 86, this is merely an example, and the reference size range 118 determined with respect to the reference value 95 may be stored in the NVM 86 and the control unit 82C may acquire the reference size range 118 from the NVM 86.

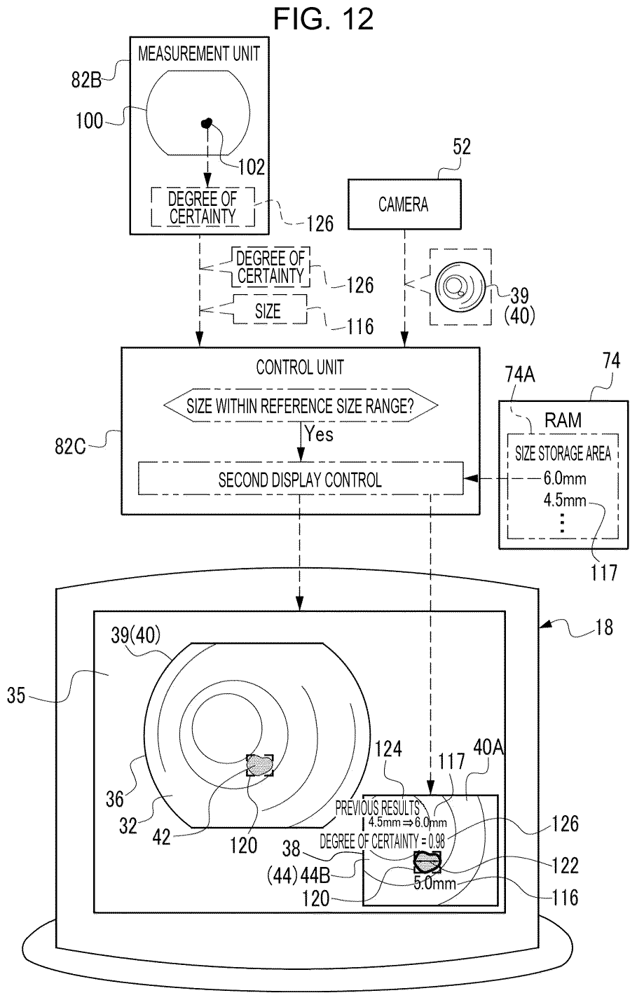

While the embodiment described above exemplifies the mean value 121 as one of the previous results 124 included in the auxiliary information 44B, the technology of the present disclosure is not limited to this. For example, as illustrated in FIG. 12, instead of the mean value 121, a degree of certainty 126 may be used as one of the previous results 124. The degree of certainty 126 is a degree of certainty (for example, a probability) assigned to the segmentation image 102 on the probability map 100 obtained by the measurement unit 82B. In the example illustrated in FIG. 12, as described above, the auxiliary information 44B displayed in the second display region 38 includes the degree of certainty 126. This allows the doctor 12 to accurately make a clinical decision on the lesion 42 by referring to the degree of certainty 126 included in the auxiliary information 44B displayed in the second display region 38. The previous results 124 may include both the degree of certainty 126 and the mean value 121. Also in this case, a similar effect can be expected.

In the example illustrated in FIG. 12, furthermore, as the information enabling the identification of the shape of the lesion 42 in the frame 40, the outer contour of the image region indicating the lesion 42 is displayed in a more highlighted manner than other image regions in the local image 40A. The outer contour of the image region indicating the lesion 42 is an example of the “shape information” according to the technology of the present disclosure. While an example embodiment in which the outer contour of the image region indicating the lesion 42 is displayed in a more highlighted manner than the other image regions is given here, this is merely an example. It is sufficient that information enabling the identification of the shape of the lesion 42 (such as the coordinates, the segmentation image 102, and/or the like) be displayed on the screen 35. In the example illustrated in FIG. 12, as described above, the outer contour of the image region indicating the lesion 42 is displayed in a more highlighted manner than the other image regions. This allows the doctor 12 to accurately make a clinical decision on the lesion 42 by referring to the outer contour of the image region indicating the lesion 42.

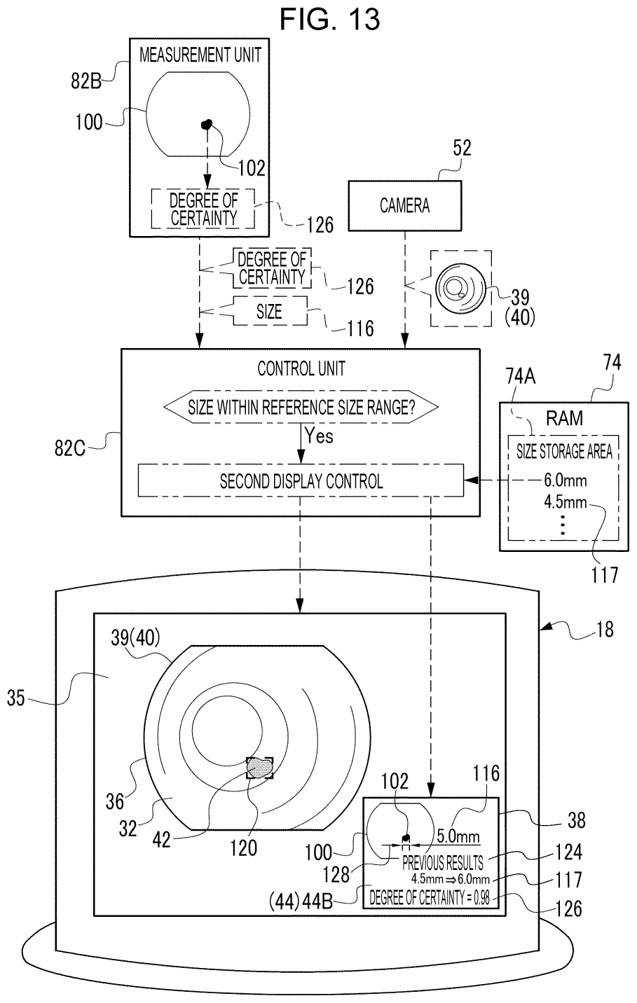

While the embodiment described above provides an example embodiment in which the auxiliary information 44B displayed in the second display region 38 includes the local image 40A, the technology of the present disclosure is not limited to this. For example, as illustrated in FIG. 13, the auxiliary information 44B displayed in the second display region 38 may include the probability map 100 in place of the local image 40A. The auxiliary information 44B displayed in the second display region 38 may include the local image 40A and the probability map 100.

For example, as illustrated in FIG. 13, the auxiliary information 44B displayed in the second display region 38 may include, together with the size 116, measurement direction information 128 that enables the identification of a measurement direction used to measure the size 116. In the example illustrated in FIG. 13, the measurement direction information 128 is assigned to the segmentation image 102 on the probability map 100. In the example illustrated in FIG. 13, a dimension line is used as an example of the measurement direction information 128. For example, an example of the dimension line used as the measurement direction information 128 is a dimension line using the line segment 110 (see FIG. 6). In the example illustrated in FIG. 13, as described above, the auxiliary information 44B displayed in the second display region 38 includes the measurement direction information 128. This allows the doctor 12 to accurately make a clinical decision on the lesion 42 by referring to the measurement direction information 128 included in the auxiliary information 44B displayed in the second display region 38.

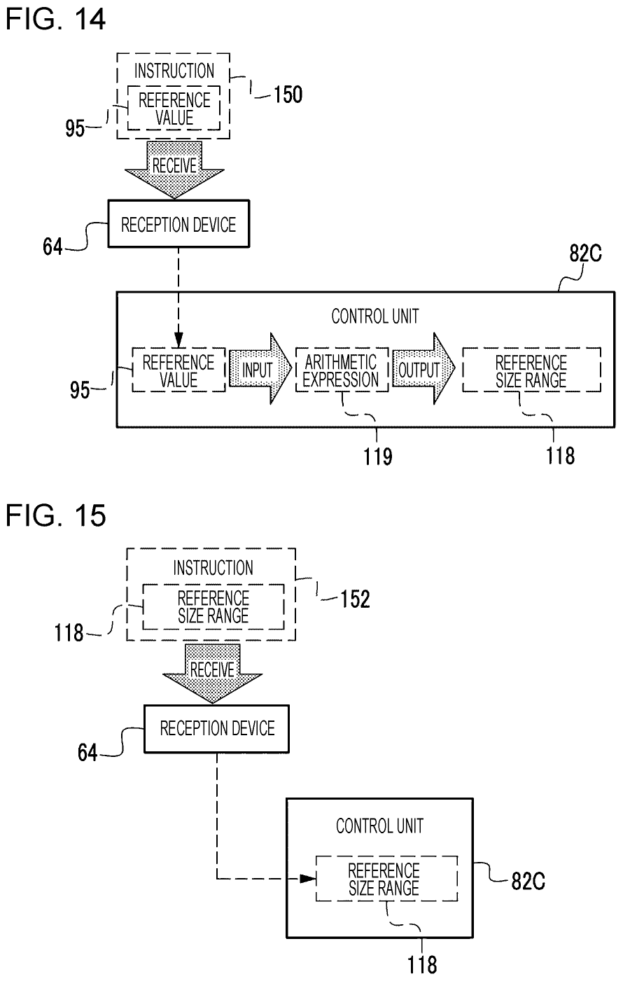

While the embodiment described above provides an example embodiment in which the reference value 95 is stored in the NVM 86, the technology of the present disclosure is not limited to this. As an example, as illustrated in FIG. 14, the reference value 95 may be determined in response to an instruction 150 given from the outside (for example, the doctor 12). In the example illustrated in FIG. 14, the instruction 150 including the reference value 95 is received by the reception device 64. Then, the control unit 82C determines the reference size range 118 in a manner similar to that in the embodiment described above, based on the reference value 95 included in the instruction 150 received by the reception device 64. In the example illustrated in FIG. 14, the instruction 150 is an example of the “instruction” according to the technology of the present disclosure.

In the example illustrated in FIG. 14, as described above, the reference value 95 is determined in accordance with the instruction 150 given from the outside. This allows the doctor 12 to make a clinical decision on the lesion 42, based on the reference value 95 determined by the doctor 12 himself/herself.

While the embodiment described above provides an example embodiment in which the reference size range 118 is determined based on the reference value 95 stored in the NVM 86, the technology of the present disclosure is not limited to this. As an example, as illustrated in FIG. 15, the reference size range 118 may be determined in response to an instruction 152 given from the outside (for example, the doctor 12). In the example illustrated in FIG. 15, the instruction 152 including the reference size range 118 is received by the reception device 64. Then, the control unit 82C acquires the reference size range 118 included in the instruction 152 received by the reception device 64. In the example illustrated in FIG. 15, the instruction 152 is an example of the “instruction” according to the technology of the present disclosure.

In the example illustrated in FIG. 15, as described above, the reference size range 118 is determined in accordance with the instruction 152 given from the outside. This allows the doctor 12 to make a clinical decision on the lesion 42, based on the reference size range 118 determined by the doctor 12 himself/herself.

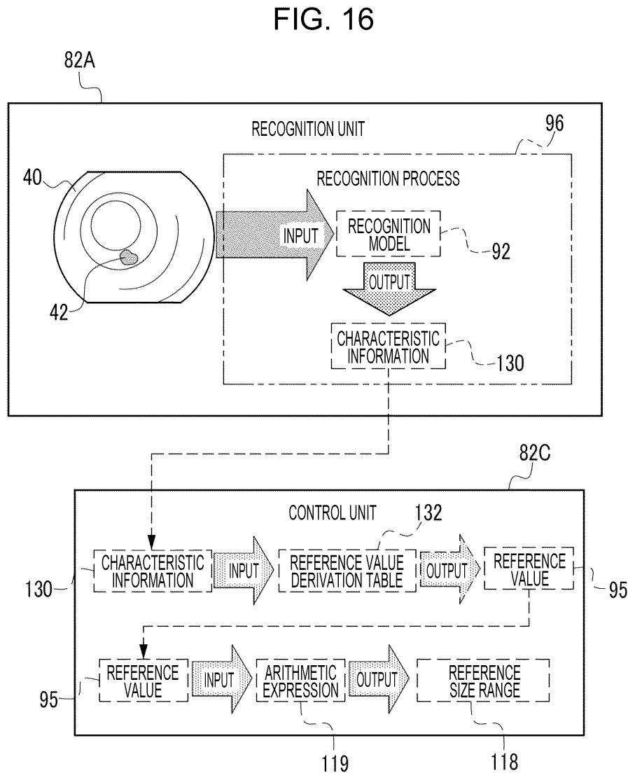

While the embodiment described above provides an example embodiment in which the reference size range 118 is determined based on the reference value 95 stored in the NVM 86, the technology of the present disclosure is not limited to this. For example, as illustrated in FIG. 16, the reference size range 118 may be determined based on characteristic information 130 output from the recognition model 92. The characteristic information 130 is information indicating the characteristics of the lesion 42 appearing in the frame 40.

An example of the characteristics of the lesion 42 is geometric characteristics of the lesion 42 (for example, the position of the lesion 42 in the frame 40, the shape of the lesion 42, and/or the size of the lesion 42), the type of the lesion 42, the category of the lesion 42, and/or the like.

The control unit 82C derives the reference value 95 using a reference value derivation table 132. The reference value derivation table 132 is a table with the characteristic information 130 as an input and the reference value 95 as an output. The control unit 82C acquires the characteristic information 130 from the recognition unit 82A and derives the reference value 95 corresponding to the acquired characteristic information 130 from the reference value derivation table 132. Then, the control unit 82C determines the reference size range 118 in a manner similar to that in the embodiment described above, based on the reference value 95 derived from the reference value derivation table 132.

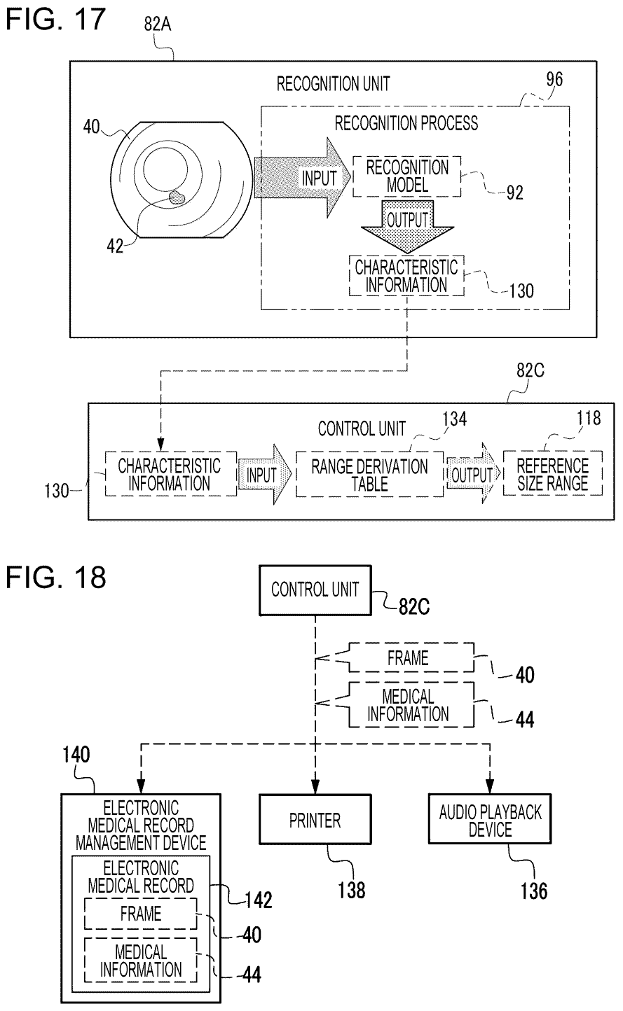

For example, as illustrated in FIG. 17, the control unit 82C may derive the reference size range 118 using a range derivation table 134. The range derivation table 134 is a table with the characteristic information 130 as an input and the reference size range 118 as an output. The control unit 82C acquires the characteristic information 130 from the recognition unit 82A and derives the reference size range 118 corresponding to the acquired characteristic information 130 from the range derivation table 134. Then, the control unit 82C determines whether the size 116 falls within the reference size range 118 derived from the range derivation table 134, and selectively performs the first display control and the second display control on the display device 18 in accordance with the determination result.

In the examples illustrated in FIGS. 16 and 17, as described above, the reference value 95 is determined based on the characteristics of the lesion 42, and the reference size range 118 is determined based on the characteristics of the lesion 42. This allows the doctor 12 to make a clinical decision based on the characteristics of the lesion 42.

While the embodiment described above provides an example embodiment in which the control unit 82C generates the distance image 106 (see FIG. 6) from the frame 40 by using the distance derivation model 94 (see FIG. 6), the technology of the present disclosure is not limited to this. For example, a depth sensor (for example, a sensor that measures a distance using a laser ranging method, a phase difference method, and/or the like) provided in the tip portion 50 (see FIG. 2) may measure the depth of the large intestine 28 in the depth direction, and the processor 82 may generate the distance image 106, based on the measured depth.

While the embodiment described above provides an example embodiment in which the endoscopic moving image 39 is displayed in the first display region 36, a result of the recognition process 96 performed on the endoscopic moving image 39 may be displayed superimposed on the endoscopic moving image 39 in the first display region 36. At least a portion of the segmentation image 102 obtained as a result of the recognition process 96 performed on the endoscopic moving image 39 may be displayed superimposed on the endoscopic moving image 39. An example in which at least a portion of the segmentation image 102 is displayed superimposed on the endoscopic moving image 39 is an example embodiment in which the outer contour of the segmentation image 102 is displayed superimposed on the endoscopic moving image 39 by using an alpha blending method.

In addition, for example, when the recognition process 96 is performed using an AI-based bounding box method, a bounding box may be displayed superimposed on the endoscopic moving image 39 in the first display region 36. Furthermore, for example, when a plurality of lesions 42 appear in the endoscopic moving image 39, at least a portion of the segmentation image 102 and/or a bounding box are displayed superimposed in the first display region 36 as information that can visually identify which of the lesions 42 the measured size 116 corresponds to. Alternatively, the probability map 100 and/or a bounding box for the lesion 42 corresponding to the measured size 116 may be displayed in a display region different from the first display region 36. Alternatively, for example, the probability map 100 may be displayed superimposed on the endoscopic moving image 39 in the first display region 36. The information displayed superimposed on the endoscopic moving image 39 may be information depicted semi-transparent (for example, information subjected to alpha blending).

While the embodiment described above provides an example embodiment in which the length, in real space, of the longest range across the lesion 42 along the line segment 110 is measured as the size 116, the technology of the present disclosure is not limited to this. For example, the length, in real space, of the range corresponding to the longest line segment parallel to the short sides of the rectangular frame 112 for the image region indicating the lesion 42 may be measured as the size 116 and displayed on the screen 35. In this case, the doctor 12 can grasp the length, in real space, of the longest range across the lesion 42 along the longest line segment parallel to the short sides of the rectangular frame 112 for the image region indicating the lesion 42.

Furthermore, the size of the lesion 42 in real space in relation to the radius and/or diameter of a circumcircle of the image region indicating the lesion 42 may be measured and displayed on the screen 35. In this case, the doctor 12 can grasp the size of the lesion 42 in real space in relation to the radius and/or diameter of the circumcircle of the image region indicating the lesion 42.

While the embodiment described above provides an example embodiment in which the size 116 is displayed in the second display region 38, this is merely an example. The size 116 may be displayed outside the second display region 38 in a pop-up manner from within the second display region 38, or the size 116 may be displayed in a region other than the second display region 38 on the screen 35. The type of lesion, the category of lesion, and/or the like may also be displayed in the first display region 36 and/or the second display region 38, or may be displayed on a screen other than the screen 35.

The embodiment described above provides an example embodiment in which the size 116 of one lesion 42 is measured and the measurement result is presented to the doctor 12. When a plurality of lesions 42 appear in the frame 40, it is sufficient that the medical support process be executed on each of the plurality of lesions 42. In this case, a mark or the like may be added to an image region of a lesion 42 corresponding to information (size, category, type, and width) displayed on the screen 35 to allow the identification of which of the lesions 42 the information displayed on the screen 35 corresponds to.

In a case where the medical support process is executed on each of the plurality of lesions 42, respective medical support process results obtained through the medical support process executed on the plurality of lesions 42 (for example, the content displayed in the second display region 38) may be displayed as a list, or may be selectively displayed in accordance with an instruction received by the reception device 64 and/or various conditions. In this case, for example, information enabling the identification of which of the lesions 42 each of the medical support process results corresponds to (for example, information in which each of the medical support process results and the corresponding one of the lesions 42 are associated with each other in a visually identifiable manner) is displayed on the screen 35.

While the embodiment described above provides an example embodiment in which the size 116 is measured frame by frame, this is merely an example, and the size 116 may be measured in units of multiple frames. Alternatively, the control unit 82C may perform a process (for example, the processes illustrated in FIGS. 9 and 10 and the like) using a representative size (for example, a mean value, a median value, a maximum value, a minimum value, a deviation, a standard deviation, a mode value, and/or the like) obtained by measuring the size 116 in units of multiple frames.

In the embodiment described above, the AI-based object recognition process is exemplified as the recognition process 96. However, the technology of the present disclosure is not limited to this. The recognition unit 82A may recognize the lesion 42 appearing in the frame 40 by the execution of a non-AI-based object recognition process (for example, template matching or the like).

While the embodiment described above describes an example embodiment in which the arithmetic expression 114 is used to calculate the size 116, the technology of the present disclosure is not limited to this. The size 116 may be measured by performing an AI-based process on the frame 40. In this case, for example, a trained model is used that, in response to an input of the frame 40 including the lesion 42, outputs the size 116 of the lesion 42. To generate the trained model, deep learning is performed on a neural network by using training data in which lesions appearing in images used as example data are assigned annotations indicating the sizes of the lesions as ground-truth data.

While the embodiment described above describes an example embodiment in which the distance information 104 is derived using the distance derivation model 94, the technology of the present disclosure is not limited to this. Other methods for deriving the distance information 104 using an AI-based method include, for example, a method for combining segmentation and depth estimation (for example, regression learning to provide the distance information 104 to the entire image (for example, all the pixels constituting the image) or unsupervised learning to learn the distance of the entire image in an unsupervised way).

While the embodiment described above exemplifies the endoscopic moving image 39, the technology of the present disclosure is not limited to this. The technology of the present disclosure is also applicable to a medical moving image (for example, a moving image obtained by a modality (for example, a radiographic diagnostic apparatus, an ultrasound diagnostic apparatus, or the like) other than the endoscope system 10, such as a radiographic moving image or an ultrasound moving image) other than the endoscopic moving image 39.

While the embodiment described above provides an example embodiment in which the size 116 of the lesion 42 appearing in a moving image is measured, this is merely an example. The technology of the present disclosure is also applicable to the measurement of the size 116 of the lesion 42 appearing in a stop-motion image or a still image.

While the embodiment described above provides an example embodiment in which the distance information 104 extracted from the segmentation-corresponding region 106A in the distance image 106 is input to the arithmetic expression 114, the technology of the present disclosure is not limited to this. For example, it is sufficient that the distance information 104 corresponding to the position identified from the position identification information 98 be extracted from among all the pieces of distance information 104 output from the distance derivation model 94, without the generation of the distance image 106, and the extracted distance information 104 be input to the arithmetic expression 114.

In the examples described above, the size 116 and the like are output to the display device 18, by way of example. However, the technology of the present disclosure is not limited to this, and various kinds of information such as the frame 40 and/or the medical information 44 (hereinafter referred to as “various kinds of information”) may be output to a device other than the display device 18. As an example, as illustrated in FIG. 18, information that can be output as audio among the various kinds of information may be output to an audio playback device 136 as a destination. The information that can be output as audio among the various kinds of information may be output as audio by the audio playback device 136. The various kinds of information may be output to a printer 138, an electronic medical record management device 140, and/or the like as a destination. The various kinds of information may be printed as text or the like on a medium (for example, a sheet) or the like by the printer 138, or may be stored in an electronic medical record 142 managed by the electronic medical record management device 140.

While the examples described above describe an example embodiment in which the various kinds of information are displayed on the screen 35 or the various kinds of information are not displayed on the screen 35, the display of the various kinds of information on the screen 35 means the display of the various kinds of information in a manner perceptible to the user or the like (for example, the doctor 12). The concept that the various kinds of information are not displayed on the screen 35 also includes a concept of reducing the display level of the various kinds of information (for example, the level perceived through the display). For example, the concept that the various kinds of information are not displayed on the screen 35 also includes a concept of displaying the various kinds of information in a display style that does not allow the user or the like to visually perceive the various kinds of information. Examples of the display style in this case include display styles in which the various kinds of information are displayed in a reduced font size, the various kinds of information are depicted as thin lines, the various kinds of information are depicted as broken lines, the various kinds of information blink, the various kinds of information are displayed for an imperceptible period of display time, and the various kinds of information are made transparent to an imperceptible level. The same applies to the various outputs described above, such as audio output, printing, and storage.

While the embodiment described above provides an example embodiment in which the medical support process is performed by the processor 82 included in the endoscope system 10, the technology of the present disclosure is not limited to this, and a device external to the endoscope system 10 may perform at least some of the processing operations included in the medical support process.

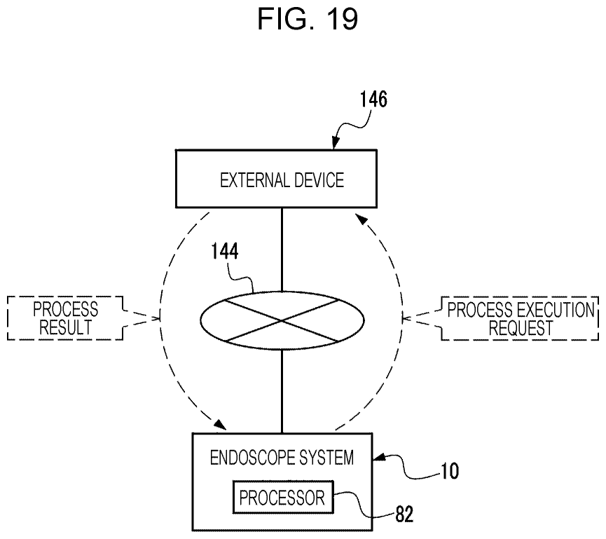

In this case, for example, as illustrated in FIG. 19, an external device 146 connected to the endoscope system 10 in a communicable manner via a network 144 (for example, a WAN, a LAN, and/or the like) is used.

Examples of the external device 146 include at least one server that transmits and receives data to and from the endoscope system 10 directly or indirectly via the network 144. The external device 146 receives a process execution instruction given from the processor 82 of the endoscope system 10 via the network 144. Then, the external device 146 executes a process corresponding to the received process execution instruction, and transmits a process result to the endoscope system 10 via the network 144. In the endoscope system 10, the processor 82 receives the process result transmitted from the external device 146 via the network 144 and executes a process using the received process result.

Examples of the process execution instruction include an instruction to cause the external device 146 to execute at least a portion of the medical support process. A first example of at least a portion of the medical support process (that is, a process to be executed by the external device 146) is the recognition process 96. In this case, the external device 146 executes the recognition process 96 in accordance with the process execution instruction given from the processor 82 of the endoscope system 10 via the network 144, and transmits a recognition process result (for example, the position identification information 98, the probability map 100, and/or the like) to the endoscope system 10 via the network 144. In the endoscope system 10, the processor 82 receives the recognition process result and executes a process similar to that in the embodiment described above by using the received recognition process result.

A second example of at least a portion of the medical support process (that is, a process to be executed by the external device 146) is a process performed by the measurement unit 82B. The process performed by the measurement unit 82B refers to, for example, a process for measuring the size 116 of the lesion 42. In this case, the external device 146 executes the process performed by the measurement unit 82B in accordance with the process execution instruction given from the processor 82 of the endoscope system 10 via the network 144, and transmits a measurement process result (for example, the size 116 or the like) to the endoscope system 10 via the network 144. In the endoscope system 10, the processor 82 receives the measurement process result and executes a process similar to that in the embodiment described above by using the received measurement process result.

A third example of at least a portion of the medical support process (that is, a process to be executed by the external device 146) is the processing of step ST22, the processing of step ST24, and/or the processing of step ST26 included in the medical support process illustrated in FIG. 11.

For example, the external device 146 is implemented by cloud computing. The cloud computing is merely an example, and the external device 146 may be implemented by network computing such as fog computing, edge computing, or grid computing. Instead of the server, at least one personal computer or the like may be used as the external device 146. Alternatively, the external device 146 may be an arithmetic device with a communication function mounted with a plurality of types of AI functions.

While the embodiment described above provides an example embodiment in which the medical support program 90 is stored in the NVM 86, the technology of the present disclosure is not limited to this. For example, the medical support program 90 may be stored in a portable non-transitory computer-readable storage medium such as an SSD or a USB memory. The medical support program 90 stored in the non-transitory storage medium is installed in the computer 78 of the endoscope system 10. The processor 82 executes the medical support process in accordance with the medical support program 90.

Alternatively, the medical support program 90 may be stored in a storage device of another computer, a server, or the like connected to the endoscope system 10 via a network, and the medical support program 90 may be downloaded in response to a request from the endoscope system 10 and installed in the computer 78.

Not all, but a portion, of the medical support program 90 may be stored in a storage device of another computer, a server device, or the like connected to the endoscope system 10, or not all, but a portion, of the medical support program 90 may be stored in the NVM 86.

Examples of a hardware resource that executes the medical support process may include the following various processors. The processors include, for example, a CPU that is a general-purpose processor configured to execute software, that is, a program, to function as a hardware resource that executes the medical support process. The processors further include, for example, a dedicated electric circuit that is a processor having a circuit configuration designed specifically for executing specific processing, such as an FPGA, a PLD, or an ASIC. Each of the processors incorporates or is connected to a memory, and uses the memory to execute the medical support process.

The hardware resource that executes the medical support process may be configured as one of the various processors or as a combination of two or more processors of the same type or different types (for example, a combination of a plurality of FPGAs or a combination of a CPU and an FPGA). The hardware resource that executes the medical support process may be a single processor.

Examples of configuring the hardware resource as a single processor include, first, a form in which a single processor is configured as a combination of one or more CPUs and software and the processor functions as a hardware resource that executes the medical support process. The examples include, second, a form in which, as typified by an SoC or the like, a processor is used in which the functions of the entire system including a plurality of hardware resources that execute the medical support process are implemented as one IC chip. As described above, the medical support process is implemented by using one or more of the various processors described above as hardware resources.

More specifically, the hardware structure of these various processors may be an electric circuit in which circuit elements such as semiconductor elements are combined. The medical support process described above is merely an example. Thus, it goes without saying that unnecessary steps may be deleted, new steps may be added, or the processing order may be changed without departing from the gist.

The description and drawings presented above provide detailed descriptions of portions according to the technology of the present disclosure and are merely examples of the technology of the present disclosure. For example, the descriptions related to the configurations, functions, operations, and effects described above are descriptions related to an example of the configurations, functions, operations, and effects of portions according to the technology of the present disclosure. Thus, it goes without saying that unnecessary portions may be deleted or new elements may be added or substituted in the description and drawings presented above without departing from the gist of the technology of the present disclosure. To avoid complexity and facilitate understanding of portions according to the technology of the present disclosure, descriptions related to common general technical knowledge and the like, for which no specific explanation is required to implement the technology of the present disclosure, are omitted in the description and drawings presented above.

As used herein, “A and/or B” is synonymous with “at least one of A or B”. That is, “A and/or B” means only A, only B, or a combination of A and B. In this specification, furthermore, a concept similar to that of “A and/or B” is applied also to the expression of three or more matters in combination with “and/or”.

All publications, patent applications, and technical standards described herein are incorporated herein by reference to the same extent as if each individual publication, patent application, and technical standard were specifically and individually indicated to be incorporated by reference.

What is claimed is:

1. A medical support device comprising a processor,
the processor being configured to:
acquire a size of an observation target region appearing in a medical image obtained by imaging an imaging target region including the observation target region with a modality; and
output auxiliary information for assisting in clinical decision making in cases in which the size falls within a size range determined with respect to a reference value for the decision making.

2. The medical support device according to claim 1, wherein
the size is measured based on the medical image.

3. The medical support device according to claim 1, wherein
the auxiliary information includes:
the size of the observation target region in at least one direction;
position information enabling identification of a position of the observation target region in the medical image;
shape information enabling identification of a shape of the observation target region;
measurement direction information enabling identification of a measurement direction used to measure the size;
a degree of certainty obtained from AI in cases in which the size is measured using the AI;
size variation range information enabling identification of a range of variation in the size identified in cases in which the size is measured based on a plurality of the medical images;
an image indicating the observation target region; and/or
a statistical measure based on previously measured size.

4. The medical support device according to claim 1, wherein
the reference value and/or the size range is determined based on medical knowledge.

5. The medical support device according to claim 1, wherein
the reference value and/or the size range is determined based on a characteristic of the observation target region.

6. The medical support device according to claim 1, wherein
the reference value and/or the size range is determined in accordance with a given instruction.

7. The medical support device according to claim 1, wherein
the auxiliary information is output by displaying the auxiliary information on a screen.

8. The medical support device according to claim 1, wherein
the auxiliary information is output by displaying the auxiliary information on a first screen,
the medical image is displayed on a second screen different from the first screen, and the first screen and the second screen are arranged in a manner that enables comparison with each other.

9. The medical support device according to claim 1, wherein
the decision making is decision making as to whether to excise the observation target region from the imaging target region.

10. The medical support device according to claim 1, wherein
the modality is an endoscope system.

11. The medical support device according to claim 1, wherein
the medical image is an endoscopic image obtained by imaging the imaging target region with an endoscope.

12. The medical support device according to claim 1, wherein
the observation target region is a lesion.

13. An endoscope system comprising:
the medical support device according to claim 1; and
an endoscope that performs imaging of the imaging target region.

14. A medical support method comprising:
acquiring a size of an observation target region appearing in a medical image obtained by imaging an imaging target region including the observation target region with a modality; and
outputting auxiliary information for assisting in clinical decision making in cases in which the size falls within a size range determined with respect to a reference value for the decision making.

15. The medical support method according to claim 14, wherein
the modality includes an endoscope, and
the medical support method comprises using the endoscope.

16. A non-transitory computer-readable storage medium storing a program executable by a computer to execute a medical support process, the medical support process comprising:
acquiring a size of an observation target region appearing in a medical image obtained by imaging an imaging target region including the observation target region with a modality; and
outputting auxiliary information for assisting in clinical decision making in cases in which the size falls within a size range determined with respect to a reference value for the decision making.
